(19) United States
(12) Patent Application Publication
(10) Pub. No.: US 2026/0152710 A1
(43) Pub. Date: Jun. 4, 2026

(54) CELL CULTURE DEVICE AND METHOD OF CULTURING CELLS

(71) Applicant: City University of Hong Kong, Hong Kong (CN)

(72) Inventors: Mengsu YANG, Hong Kong (CN); Zhengdong ZHOU, Hong Kong (CN); Xiaoyu ZHOU, Hong Kong (CN); Wenxiu LI, Hong Kong (CN); Jiayu SUN, Hong Kong (CN)

(21) Appl. No.: 18/964,768

(22) Filed: Dec. 2, 2024

(51) Int. Cl. C12M 1/00; C12M 1/12; C12M 1/32; C12M 1/34; C12N 5/00

(52) U.S. Cl. CPC C12M 23/34; C12M 23/12; C12M 25/14; C12M 41/46; C12N 5/0068; C12N 2533/54

(57) ABSTRACT

A cell culture device including: a top component, wherein the top component includes a cell culture chamber and one or more cell culture medium reservoirs; wherein the cell culture chamber includes an outer area and an inner area, wherein the outer area and inner area of the cell culture chamber are separated from each other by at least one row of micropillars, and wherein the outer area of the cell culture chamber is fluidly connected to the one or more cell culture medium reservoirs, and wherein the cell culture chamber includes an inlet which allows addition of a mixture including cells to the inner area of the cell culture chamber; a method of use thereof; and a system including the same.

### TECHNICAL FIELD

The present disclosure relates to an apparatus for microbiology and tissue culture. The disclosure also relates to a device for cell culture and method of culturing cells using the same for creating tumor microenvironment gradient in a two-dimensional plane.

### BACKGROUND

Three-dimensional (3D) tumor spheroid models provide a more faithful representation of in vivo tumor physiological characteristics and complexities, compared to conventional two-dimensional (2D) culture systems. A tumor spheroid encompasses spatial gradient distribution characteristics, which is also a hallmark of a solid tumor. This spatial variation is shaped by the intricate interplay between vascular permeability and the robust proliferative capacity of tumor cells, resulting in a gradient environment ranging from regions proximal to blood vessels to distal areas. The gradient microenvironment has been recognized for its critical role in the progression of tumorigenesis, invasion, metastasis, and the acquisition of drug resistance.

As the tumor spheroid grows, the augmented distance between the inner core of the tumor spheroid and the exterior microenvironment gives rise to the establishment of chemical gradients, such pH, oxygen, glucose, ATP distribution, lactate accumulation gradients, thereby impeding the metabolic activities of the tumor cells in the internal space. As a result, distinct regions with varying proliferative capacities emerge, classifying a spheroid into a peripheral proliferating layer, an intermediate quiescent layer, and an inner necrotic core. Benefiting from this similarity, research based on tumor spheroids has achieved significant progress, greatly enhanced our understanding of the biology of a tumor and contributed to therapeutic advancements.

However, in vitro tumor spheroid models comprise two limitations inherent to their characteristics, primarily concerning size and shape. In context to the former, research indicates that as spheroids grow, solid stress induced by cell growth and proliferation imposes constraints to their growth, with an upper diameter limit estimated at 1000 μm or 1 mm. At this scale, downstream analysis, such as paraffin embedding of the tumor spheroid and sectioning of the tumor spheroid preparation, becomes challenging, particularly making the isolation of cells from different proliferation regions for molecular analysis extremely difficult. The second challenge lies in their spherical shape. Internal features of the spheroids are hard capture and monitor in real-time using conventional inverted fluorescence microscopy. Typically, internal features of the spheroids are captured and monitored using scanning confocal microscopy through optical sectioning.

Advancements in microfluidic technology have addressed several challenges faced within traditional tumor spheroid models. Different microfluidic devices have been developed to mimic and control gradient characteristics, such as oxygen, pH, cell viability of tumor spheroids or solid tumor primarily utilizing flow-based or diffusion-based strategies. The vast majority of microfluidic platforms utilize transparent substrates, simplifying real-time observation of different microenvironmental regions.

However, a device that establishes the spatial distribution characteristics of a tumor spheroid, characterized by radial distribution from the peripheral layer to the inner core, and subsequently allows for the extraction of cellular components for molecular analysis such as histological staining, qPCR analysis or sequencing, has not yet been disclosed. Therefore, it is the object of the present disclosure to provide a device to replicate both the phenotypic and physiological gradient features of a tumor spheroid, so as to facilitate the elucidation of the influence of the gradient microenvironment on the biology of a tumor.

### SUMMARY

In one aspect, the present disclosure relates to a cell culture device comprising a top component, wherein the top component comprises a cell culture chamber and one or more cell culture medium reservoirs; wherein the cell culture chamber comprises an outer area and an inner area, wherein the outer area and inner area of the cell culture chamber are separated from each other by at least one row of micropillars, and wherein the outer area of the cell culture chamber is fluidly connected to the one or more cell culture medium reservoirs, and wherein the cell culture chamber comprises an inlet which allows addition of a mixture comprising cells to the inner area of the cell culture chamber.

In another aspect, the present disclosure relates to a method for culturing cells using the cell culture device as disclosed herein.

In another aspect, the present disclosure relates a system comprising: a. the cell culture device as disclosed herein; b. a multi-well cell culture plate; and c. a detection device for detecting signals emitted from cells cultured in the cell culture device.

### BRIEF DESCRIPTION OF THE DRAWINGS

FIG. 1 is an illustration of the tumor microenvironment and an exemplary design of the cell culture device. (A) shows the similarity of the gradient microenvironmental features between a tumor spheroid and the tumor microenvironment. Both the tumor spheroid and tumor microenvironment are characterized by gradients including, but not limited to oxygen, pH and nutrients availability. The classical tumor spheroid exhibits classic features, including a peripheral proliferative area, an intermediate quiescent layer, and an inner necrotic core. (B) is an exemplary schematic of the workflow of culturing tumor cells in an exemplary cell culture device as disclosed herein to obtain a cell aggregate characterized by the tumor microenvironmental gradient. Firstly, cells mixed with a scaffold, such as gelatin methacrylate (GelMA) were loaded into the cell culture chamber of the cell culture device through a central inlet. Once the cells and GelMA mixture is solidified, the inlet was sealed with a thin glass cover. A suitable cell culture medium was added into the medium reservoirs of the device. The cell culture device loaded with the cells and GelMA mixture and filled with a suitable cell culture medium was subsequently placed inside the well of a multi-well plate and incubated under standard cell culture conditions for gradient microenvironment formation. The purpose of placing the cell culture device into the well of a multi-well plate is to facilitate routine medium changes and regular microscopic imaging. (C) is the technical drawing an example of the design and the dimensions of the top component (1) of the cell culture device, which, in this example, is made of polydimethylsiloxane (PDMS). The top view, front cross section view, side cross section view and zoomed-in top view are shown in the present figure. (1): top component of the cell culture device; (10): cell culture chamber; (101): inner area of the cell culture chamber; (102): an inlet or opening for loading of a sample or mixture comprising cells; (103): outer area of the cell culture chamber, which is fluidically connected with two cell culture reservoirs; (104): rows of micropillars separating the outer and inner area of the cell culture chamber; (105): micropillars arranged in a staggered fashion; (106): channel leading to the inner area of the cell culture chamber with the inlet on one end and the cell culture chamber on the other end; (107): micropillars inside the inner area of the cell culture chamber, which are sparsely arranged; (11): cell culture reservoir. (D) shows the exemplary fabrication and assembly of the cell culture device, with the top component (1) attached to a round glass substrate (2). The cell culture device is assembled by bonding the top component (1) with the bottom glass substrate (2). Once assembled, the device can be placed inside the well a multi-well plate for cell culture. The top component (1) and bottom substrate (2) can be disassembled by detaching the top component (1) and bottom substrate (2) after the experiment to retrieve cells grown on the bottom substrate (2). The top view of the assembled device which was filled with cresyl violet solution is shown in the right panel in (D). As can be seen in from the top view of the cell culture device in the right panel of (D), the top component (1) and bottom substrate (2) were bonded together to form a functional the cell culture device. Cresyl violet solution was added to the cell culture medium reservoirs (11) to demonstrate the distribution of the culture medium in the cell culture device. The outer area of the central cell culture chamber (103) is fluidically connected with the two cell culture reservoirs (11), one on the opposite side of the cell culture chamber (10). The outer area (103) and inner area (101) are separated by the micropillars such that the cell culture medium only comes into contact directly with the cells in the periphery of the inner area (101). The inlet (102) for loading of sample into the cell culture chamber (10) is in the centre of the cell culture chamber. (E) is a side view of the assembled cell culture device comprising the top component (1) that is bonded to a bottom substrate (2) that had been loaded with a cell mixture and cell culture medium. The cell mixture was loaded into the cell culture chamber via the inlet (102). The inlet (102) leads to the inner area of the cell culture chamber (101) via a channel (106). The cell culture reservoirs (11), which are fluidically connected to the outer area of the cell culture chamber (103), is filled with a cell culture medium, so that the cell culture medium flows into the outer around of the cell culture chamber (103) to surround the inner area of the cell culture chamber (101). (F) describes the steps of culturing cells using the cell culture device. The steps include: (a) loading a cell mixture into the cell culture device through the inlet (102) into the channel (106) which leads to the inner area of the cell culture chamber (101); (b) adding a cell culture medium into the cell culture reservoirs (11) and (c) sealing the inlet (102) by placing a small glass cover over the inlet (102).

FIG. 2 shows the phenotype of the spheroid-like gradient microenvironment in the cell aggregate obtained from culturing tumor cells in the cell culture device as disclosed herein. The tumor cells used in the experiment are SK-Hep-1 cells transfected with green fluorescent protein (GFP). GFP transfected SK-Hep-1 cells were cultured on the cell culture device to obtain the cell aggregate. (A) shows the gradient distribution of tumor cells across the cell aggregate, with the proliferative layer in the outer layer showing highest fluorescent signal and the inner necrotic core showing low to no fluorescent signal. The scale bar on the first panel from the left and the middle panel is 1000 μm and 200 μm, respectively. (B) shows the phenotype and relative diameter of spheroids cultured in suspension plates with varying sizes (spheroid 1-7) against the cell aggregate obtained from the chamber of the cell culture device as disclosed herein (spheroid 8). The scale bar on the left panel is 1000 μm. Fluorescence images captured using a common fluorescence microscope of an exemplary spheroid and exemplary cell aggregates are shown in (C) and (D), respectively. The scale bars on (C) are 200 μm and the scale bar on the left panel of (D) is 1000 μm. The distribution of fluorescence intensity of GFP in the cell aggregate from the centre (0 um) to the peripheral of the cell aggregate is shown in the graph in (D), indicating that the GFP signal is the most intense in the peripheral and the lowest in the inner core.

FIG. 3 demonstrates that culturing cells in the cell culture device with a higher initial density of cells accelerates the formation of the gradient microenvironment. (A) are photomicrographs of cell aggregates obtained from culturing SK-Hep-1 cells transfected with GFP in the cell culture device with different density of cells ranging from about 3.124×10⁴ cells to about 1×10⁶ cells in 10 μL of 10% gelatin methacrylate (GelMA). As can be seen in (A), loading the cells-GelMA mixture at a density of 1×10⁶ cells per 10 μL GelMA resulted in the obvious appearance of gradient characteristics from Day 4. However, with decreased cell densities of 5×10⁵ cells per 10 μL GelMA and 2.5×10⁵ cells per 10 μL GelMA, gradient features only began to appear from day 6 and day 14, respectively. Cell aggregates obtained from initial cell densities lower than 2.5×10⁵ cells per 10 μL GelMA formed the gradient microenvironment only at the end of the monitored 14 days. As the cells were loaded into the device through the cell loading inlet (2), a bright point is formed in the centre of the cell aggregate in the earlier time points due to cell residue in the channel (6) and light refraction. Scale bars=1000 μm. (B) shows (I-VI) the quantification of fluorescence intensity of the cell aggregates in (A), corresponding to the regions from the edge to the centre of the cell aggregates. The trend in fluorescence intensity over time indicates that as the starting density of cells in the cell culture device decreases, nutrient consumption rate gradually falls below diffusion rate, making it difficult to establish the gradient environment.

FIG. 4 shows the gradient distribution features of pH, hypoxic profile and stiffness of cell aggregates cultured in the cell culture device. Three commercially available live-staining probes were used to characterize the gradient features of the cell aggregate formed from culturing SK-Hep-1 cells in the cell culture device. The SK-Hep-1 cells were pre-incubated with the respective dyes, and then mixed with cells which were not incubated with the dyes, before being loaded and cultured in the cell culture device. Imaging was performed after 8 hours of culture to assess the gradient conditions at the early stage of the cell mixture cultured in the inner area of the cell culture chamber (101). The pH distribution status was evaluated using pHrodo Red AM or BCECF AM. (A) shows imaging of pHrodo Red AM, and a gradient of increased fluorescence signal is detected from the periphery toward the central region, indicating that the central region had a more acidic microenvironment profile compared to the periphery. Using another probe, BCECF AM, it is also confirmed that there was a decrease in fluorescence signal from the periphery toward the central area of the cell aggregate, indicating that the pH in the central area is lower than the periphery (B). Culturing cells pre-stained with the hypoxia probe, Image-iT Green, confirmed that the cells in the central area of the cell aggregate were in severe hypoxic condition, which was gradually alleviated towards the outer regions (C). After seven days of culture, wherein the gradient phenotype had clearly been formed, cell aggregates were exposed, by disassembling the top PDMS component from the bottom substrate, for stiffness testing. Stiffness distribution features of the cell aggregate were analysed by atomic force microscopy (AFM) and the analysis of stiffness profile showed that the central region of the cell aggregate is stiffer than the peripheral of the cell aggregate (see (D); ****p<0.0001). Scale bars=1000 μm

FIG. 5 shows the ability of external substance to penetrate into the central region of a spheroid-like environment is reduced with the formation of a gradient microenvironment. In this experiment, rhodamine was used to measure the ability of a substance to penetrate across three distinct microenvironments. Specifically, the first microenvironment, “Chip_gel” represents the microenvironment when the cell culture device was loaded only with gelatin methacrylate (GelMA) without cells. The second microenvironment, “Chip_day0” represents the microenvironment formed when the cell culture device is loaded with GelMA-cell mixture prior to in vitro culture. The third microenvironment, “Chip_env” represents the microenvironment formed when the cell culture device was loaded with GelMA-cell mixture and cultured for 10 days, thus representing a fully established gradient microenvironment. In the Chip_gel microenvironment permeation of rhodamine can be seen in the internal regions of the cell culture chamber within approximately 2 hours (A and B). The mean diffusion speed of rhodamine in the Chip_gel microenvironment was approximately 20.7±7.3 μm/min (C). In the Chip_day0 microenvironment, wherein the tumor microenvironment within the device had not yet been established, the presence of cells alone significantly reduced the penetration rate of rhodamine (D and E) as compared to the Chip_gel microenvironment. The penetration speed of rhodamine in the Chip_day 0 microenvironment is around 4.0±2.4 μm/min (F). The introduction of rhodamine into Chip_env microenvironment, wherein a gradient microenvironment had been established, revealed that rhodamine penetration is the slowest. External rhodamine in the Chip_env microenvironment took a longer time to penetrate into the central regions of the GelMA-cell mixture (G and H). The overall diffusion speed was significantly slower at 1.53±0.2 μm/min (see I) as compared to the other two microenvironments. Towards the end of the monitoring period, the penetration rate of rhodamine in the Chip_env had nearly reached zero, indicating that formation of a gradient microenvironment impedes the penetration of external substances into the central region of the cell aggregate. Scale bars=1000 μm

FIG. 6 shows the device's disassembly ability for releasing the cell aggregate from the cell culture device for downstream histological and molecular analysis. (A) is a schematic to illustrate the disassembly of the cell culture device, removal of the cell aggregate, and subsequent processing of the cell aggregate for histology sectioning and other staining procedures. (B) are photomicrographs showing Haematoxylin and Eosin (H&E) stained sections of different regions of the cell aggregate that had been cultured for 7 days. Scale bars are 500 μm in the first panel on the left, and 100 μm in the second, third and fourth panels from the left, respectively. Shrinkage during histological processing and the position of the section within the cell aggregates contribute to the reduced size observed in the final images. (C) are photomicrographs of immunofluorescence staining of cytokeratin and N-cadherin performed on the cell aggregate. Scale bars are 1000 μm in the first panel on the left and 50μm in the second, third and fourth panels from the left, respectively. (D) shows the procedures for retrieving cells from the inner, middle and outer region of the cell aggregate using punches of different sizes, corresponding to the inner core, intermediate layer and peripheral layer of a tumor spheroid, respectively. The retrieved cell aggregates were digested with collagenase to obtain cell suspensions for downstream analysis, such as qPCR. (E) shows the gene expression levels of various cell cycle related genes from the cells obtained from the peripheral layer, intermediate layer and inner core of the cell aggregate. There is a noticeable decrease in the expression of CCNA1, CCNB1, CCND1, CCNE1, CDC42, CDK1, CDK2, NUPF2, SETD5 and TMEM241 from the outer to the inner regions, indicating cellular arrest in the quiescent and necrotic inner areas.

### DETAILED DESCRIPTION

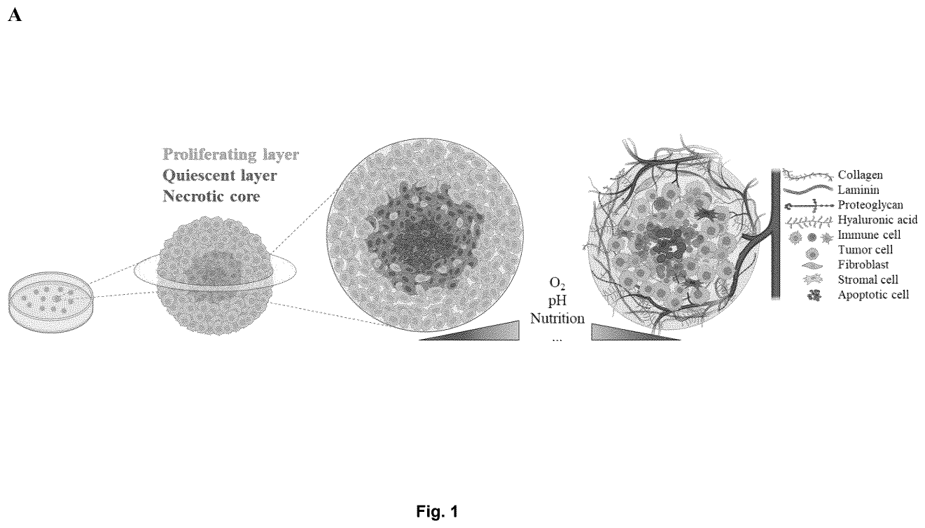

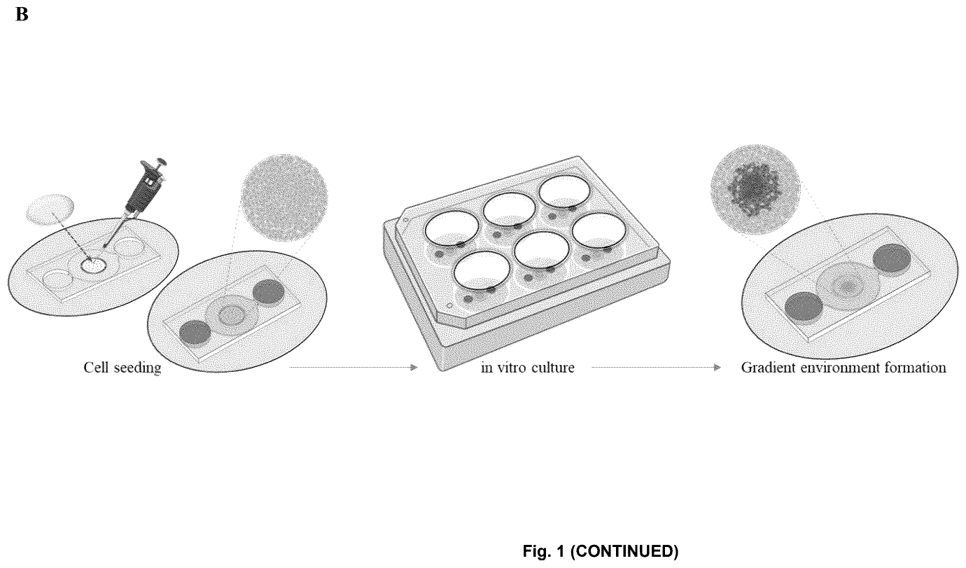

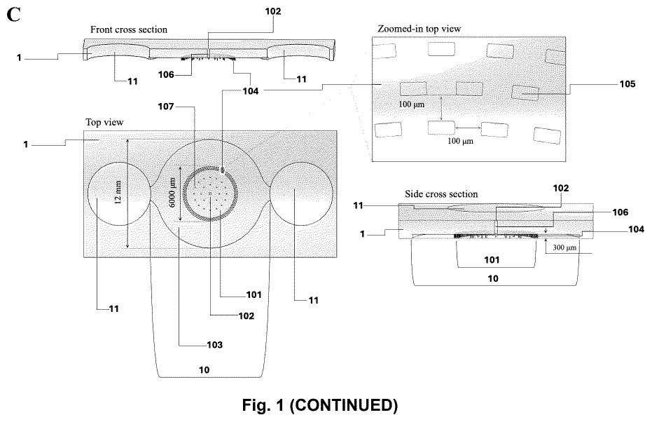

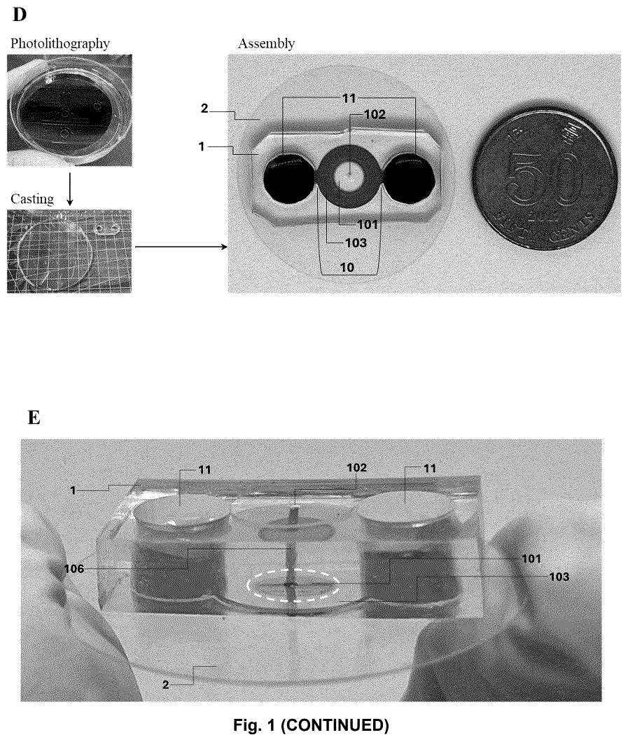

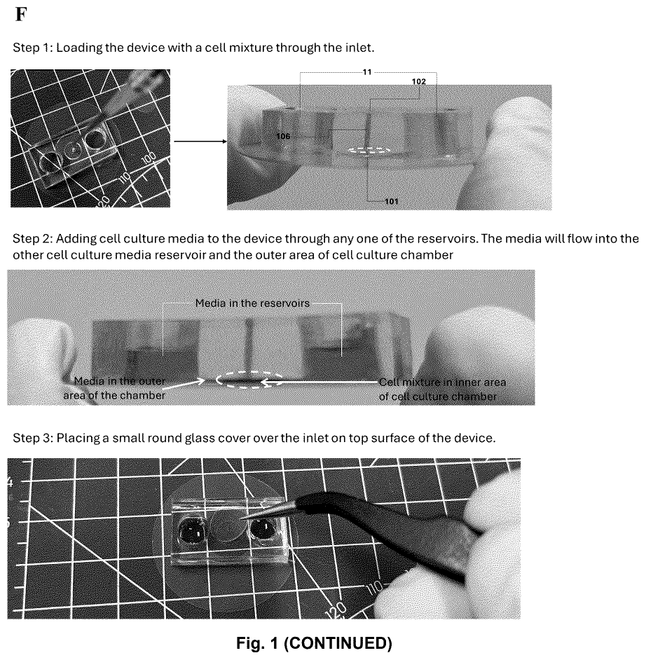

The present disclosure provides a cell culture device for projecting essential features of a 3D tumor spheroid onto a 2D plane without requiring complex tubing systems. The design of the cell culture device is based on the mechanism of formation of tumor spheroids, which is a closed spherical structure that enables the diffusion of nutrients from the periphery to the inner core (FIG. 1A). The inlet for loading cells is positioned at the center of the cell culture chamber of the cell culture device as disclosed herein which facilitates the establishment of a circular, closed, and continuous gradient microenvironment, resulting in the edge of the cell culture chamber forming a continuous and circular contour. Consequently, this allows for a concentric diffusion process from the uninterrupted periphery towards the center, effectively replicating the permeation and penetration characteristics of substances entering the tumor spheroid.

Therefore, in one aspect, the present disclosure refers to a cell culture device comprising a top component (1) wherein the top component (1) of the cell culture device comprises a cell culture chamber (10) and one or more cell culture medium reservoirs (11); wherein the cell culture chamber comprises an outer area (103) and an inner area (101); wherein the outer area and inner area of the cell culture chamber are separated from each other by at least one row of micropillars (104), and wherein the outer area (103) of the cell culture chamber is fluidically connected to the one or more cell culture medium reservoirs (11); wherein the cell culture chamber comprises an inlet (102) which allows addition of a mixture comprising cells to the inner area (101). As used herein, the term “fluidically connected” or “fluidly connected” means that fluid, such as, but not limited to, a cell culture medium, can flow from the at least one cell culture reservoir to the outer area of the cell culture chamber.

In one example, the cell culture chamber further comprises a bottom substrate (2). In another example, the top component (1) and bottom substrate (2) of the cell culture device are detachably attached. As used herein, the term “detachably attached” or “detachably attachable” refers to the simultaneous ability of the top component (1) and bottom substrate (2) of the cell culture device to attach to each other and to be detached from each other at the end of the cell culture experiment to dissemble the cell culture device to retrieve the cells cultured on the device. The top component (1) and the bottom substrate (2) are joined together or attached to each other to form a usable cell culture device.

As understood by a person skilled in the art, the top component (1) and bottom substrate (2) can be attached or joined together via different means. In one example, the top component and bottom substrate are bonded together. As used herein, the term “bonded together” refers to attachment of two surfaces that have been joined together, by means of an adhesive substance, heat or pressure. Thus, in another example, the top component is bonded to the bottom substrate using uncured polydimethylsiloxane (PDMS). The uncured PDMS acts as an adhesive to bond the top component (1) and bottom substrate (2) together. When the uncured PDMS is heated and cured, the top component (1) becomes bonded to the bottom substrate (2). In another example, the top component (1) and bottom substrate (2) can be bonded together using plasma bonding method. In this case, both top component (1) and bottom substrate (2) are subjected to plasma treatment such that the plasma treatment chemically modifies the surfaces of the top component (1) and the bottom substrate (2) allowing them to bond together tightly upon contact. The process to disassemble the top component (1) and the bottom substrate involves gently sliding a thin blade along the contact surface between the top component (1) and the bottom substrate (2) to separate them, taking care not to disturb the cell aggregate on the bottom substrate (2).

As disclosed herein, the top component (1) of the cell culture device is fabricated from polydimethylsiloxane (PDMS) using photolithographic procedures known in the art. The top component can also be produced using 3D-printing technology. As can be appreciated by a person skilled in the art, the thickness or the height of the top component (1) varies based on the volume of PDMS used during the fabrication or production process. Therefore, pouring a larger volume of PDMS during the molding process will produce in a thicker top component (1), while pouring a smaller volume of PDMS will yield a thinner top component (1). However, regardless of the thickness of the top component (1), the depth of the cell culture chamber (10), which is determined by the height of the micropillars (104), remains constant. In other words, the thickness of the top component (1) per se does not significantly affect the actual cell culture process. However, if the top component is too thin, the at least one cell culture medium reservoirs (11) will be too shallow to hold sufficient culture medium, which may negatively impact the cells growing in the cell culture chamber. Conversely, if the top component (1) is too thick, the channel (106) connecting the inlet (102) to the inner area (101) of the cell culture chamber will also become longer, potentially increasing fluorescence signal interference in the central area of the cell culture chamber, which is undesirable. The optimum thickness of the top component (1) is therefore balance between providing enough depth for the at least one cell culture medium reservoir (11) and reducing the length of the channel (106) to reduce fluorescence signal interference in the central area of the cell culture chamber.

Therefore, in one example, the thickness of the top component (1) is about 5 mm to about 10 mm. In another example, the thickness of the top component (1) is about 6 mm to about 8 mm. In some examples, the thickness of the top component (1) is about 5 mm, about 6 mm, about 7 mm, about 8 mm, about 9 mm or about 10 mm.

When the top component (1) is attached to the bottom substrate (2), a functional cell culture device comprising a cell culture chamber (10) and at least one cell culture medium reservoir (11) is formed. The cell culture chamber (10) defines the area in the cell culture device as disclosed, wherein the cells are loaded and cultured. In one example, the cell culture chamber comprises an outer area (103) and an inner area (101). The cell culture chamber can have a rectangular, squarish or circular shape. The shape and size of the cell culture chamber can be adapted depending on the desired application and type of cells to be cultured in the cell culture chamber. In one example, the volume of the cell culture chamber is about 20 mm³ to about 60 mm³. In one example, the volume of the cell culture chamber is about 30 mm³ to about 50 mm³. In one example, the volume of the cell culture chamber is about 35 mm³ to 45 mm³. In some examples, the volume of the cell culture chamber is about 20 mm³, about 25 mm³, about 32 mm³, about 34 mm³, about 35 mm³, about 36 mm³, about 38 mm³, about 40 mm³, about 45 mm³, about 50 mm³, about 55 mm³ or about 60 mm³.

In a preferred example, the cell culture chamber (10) is circular. In another example, the cell culture chamber (10) has a diameter of about 10 mm to about 30 mm. In another example, the cell culture chamber (10) has a diameter of about 15 mm to about 25 mm. In another example, the cell culture chamber (10) has a diameter of about 18 mm to about 22 mm. In some examples, the cell culture chamber (10) of the cell culture device as disclosed herein has a diameter of about 10 mm, about 11 mm, about 12 mm, about 13 mm, about 14 mm or about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm or about 30 mm. In a preferred example, the cell culture chamber (10) has a diameter of about 12 mm.

In another example, the cell culture chamber (10) comprises an inner area (101) and an outer area (103). The inner area (101) of the cell culture chamber defines the area wherein cells are loaded and cultured. In one example, the inner area (101) of the cell culture chamber has a diameter of about 4 mm to about 10 mm. In another example, the inner area (101) of the cell culture chamber has a diameter of about 5 mm to about 9 mm. In another example, the inner area (101) of the cell culture chamber has a diameter of about 6 mm to about 8 mm. In another example, the inner area (101) of the cell culture chamber has a diameter of about 4 mm, about 5 mm, about 6 mm, about 7 mm, about 8 mm, about 9 mm or about 10 mm. In a preferred example, the diameter of the inner area (101) of the cell culture chamber is about 6 mm.

The cell culture chamber (10) can be positioned in any position within the top component (1) of the cell culture device. In one example, the cell culture chamber (10) is positioned in the centre of the top component (1) of the cell culture device. An example of the top component (1) of the cell culture device is shown in FIG. 1C, wherein the cell culture chamber (10) is circular, having an inner area (101) of about 6 mm in diameter and is in located the centre of the top component (1) of the cell culture device.

The depth of the cell culture chamber (10) is determined by the height of the micropillars separating the inner area (101) and outer area (103) of the cell culture chamber. In one example, the cell culture chamber (10) has a depth of about 100 μm to about 500 μm. In another example, the depth of the cell culture chamber (10) is about 200 μm to about 400 μm. In another example, the depth of the cell culture chamber (10) is about 250 μm to about 350 μm. In another example, the cell culture chamber (10) has a depth of about 270 μm to about 330 μm. In another example, the cell culture chamber (10) has a depth of about 280 μm to about 320 μm. In another example, the cell culture chamber (10) has a depth of about 100 μm, about 150 μm, about 200 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380, about 390 μm μm, about 400 μm, about 450 μm or about 500 μm. In another example, the cell culture chamber (10) has a depth of about 300 μm. In a preferred example, the cell culture chamber (10) comprises an inner area (101) of about 6 mm in diameter and about 300 μm in depth.

The volume of the inner area (101) of the cell culture chamber (10) is dependent on the width and the depth of inner area (101) of the cell culture chamber. Therefore, in one example, the volume of the inner area (101) of the cell culture chamber is about 3 mm³ to 25 mm³. In another example, the volume of the inner area (101) of the cell culture chamber is about 5 mm³ to 20 mm³. In another example, the volume of the inner area (101) of the cell culture chamber is about 9 mm³ to 15 mm³. In another example, the volume of the inner area (101) of the cell culture chamber is about 10 mm³ to 12 mm³. In some examples, the inner area (101) of the cell culture chamber is about 3 mm³, about 4 mm³, about 5 mm³, about 6 mm³, about 7 mm³, about 8 mm³, about 9 mm³, about 10 mm³, about 11 mm³, about 12 mm³, about 13 mm³, about 14 mm³, about 15 mm³, about 16 mm³, about 17 mm³, about 18 mm³, about 19 mm³, about 20 mm³, about 21 mm³, about 22 mm³, about 23 mm³, about 24 mm³ or about 25 mm³.

To facilitate loading of samples into the cell culture device as disclosed herein, in the top component (1) of the cell culture device as disclosed herein comprises a sample or cell loading inlet (102), which is an opening formed by creating a channel (106) through the top component (1) of the cell culture device. Thus, the inlet (102) on the surface leads to the inner area (101) of the cell culture chamber via the channel (106). As disclosed herein, the terms “inlet” and “opening” are used interchangeably. In some instances, the inlet is also referred to as “cell loading inlet” or “surface inlet”. The inlet is (102) on one end of the channel (106) and the inner area (101) of the cell culture chamber is on the opposite end or the other end of the channel (106). The sample or cells loaded via the inlet (102) enters and fills the inner area (101) of the cell culture chamber via the channel (106). The size of the opening or inlet (102) can be selected depending on the application and the cell mixture to be injected into the inner area (101) of the cell culture chamber through the loading inlet (102). In one example, the inlet (102) is about 0.2 mm to about 3 mm in diameter. In another example, the inlet (102) is about 0.5 mm to about 1 mm. In another example, the inlet (102) is about 0.6 mm to about 0.8 mm. In another example, the cell loading inlet (102) is about 0.5 mm, about 0.6 mm, about 0.7 mm, about 0.8 mm about 0.9 mm, about 1.0 mm, about 1.2 mm, about 1.4 mm, about 1.6 mm, about 1.8 mm, about 2 mm, about 2.2 mm, about 2.4 mm about 2.6 mm about 2.8 mm or about 3 mm. In one example, the inlet (102) is about 0.5 mm. In another example, the inlet (102) is about 1.0 mm.

In a preferred example, the cell culture chamber (10) of the cell culture device as disclosed herein has a depth of 300 μm, wherein the inner area (101) of the cell culture chamber is about 6 mm and wherein the size of the opening for sample or cell loading (102) is about 1 mm. In one example, the cell loading inlet (102) is aligned to the centre of the inner area (101) of the cell culture chamber (10) as shown in FIG. 1C. In another example, the inlet (102) is closable. The inlet (102) can be closed or sealed to prevent oxygen from entering the cell culture chamber (10). In one example, the inlet (102) can be closed by placing a glass cover over the inlet (102).

As described herein, the cell culture chamber (10) of the cell culture device as disclosed herein comprises an outer area (103) and an inner area (101), the outer area (103) and inner area (101) are separated from each other by at least one row of micropillars (104). As described herein, the height of the micropillars (104) can determine the depth of the cell culture chamber (10). In one example, the height of the micropillars (104) is about 100 μm to about 500 μm. In another example, the height of the micropillars (104) is about 200 μm to about 400 μm or about 250 μm to about 350 μm. In another example, the height of the micropillars (104) is about 270 μm to about 330 μm. In another example, the height of the micropillars (104) is about 280 μm to about 320 μm. In another example, the height of the micropillars (104) is about 100 μm, about 150 μm, about 200 μm, about 250 μm, about 260 μm, about 270 μm, about 280 μm, about 290 μm, about 300 μm, about 310 μm, about 320 μm, about 330 μm, about 340 μm, or about 350 μm, about 360 μm, about 370 μm, about 380, about 390 μm, about 400 μm, about 450 μm or about 500 μm. In another example, the height of the micropillars (104) is about 300 μm.

The micropillars (104) can define the shape or configuration of the inner area of the cell culture chamber (101) depending on the arrangement of the micropillars (105). For example, the micropillars can be arranged to define a rectangular, squarish or circular inner area. The size of the inner area (101) of the cell culture chamber is determined by the boundary set by the micropillars (104). Therefore, in one example, the at least one row of micropillars (104) is arranged to produce a circular inner area (101) diameter of about 4 mm to about 10 mm. In another example, the at least one row of micropillars (104) is arranged to produce an inner area (101) diameter of about 5 mm to 9 mm. In another example, the at least one row of micropillars (104) is arranged to produce an inner area (101) diameter of about 6 mm to 8 mm. In some examples, the at least one row of micropillars (104) is arranged to produce an inner area (101) diameter of about 4 mm, about 5 mm, about 6 mm, about 7 mm, about 8 mm, about 9 mm or about 10 mm. In a preferred example, the at least one row of micropillars (104) are arranged to produce an inner area (101) diameter of about 6 mm.

The functions of the micropillars (104 and 105) are to prevent leakage of the sample of cell mixture during initial loading and to allow the cell culture medium to flow into the outer area (103) of the cell culture chamber (10) to surround the edge of the inner area (101) of the cell culture chamber as shown in FIG. 1D. Preventing leakage means that the cells introduced into the inner area (101) should stay in the inner area (101) and should not be able to grow out of the inner area (101) into the outer area (103) or flow out of the inner area (101) into the outer area (103) of the cell culture chamber during the initial loading of the cell mixture into the inner area (101). The number of rows of micropillars (105) is selected to ensure that the cells in the cell mixture, which is loaded into the inner area (101) of the cell culture chamber, do not leak out of the inner area (101). Another criterion for the selection of the number of rows of micropillars (105) is that the arrangement of the rows of micropillars should allow the nutrients in the cell culture medium from the fluidically connected cell culture medium reservoirs (11) to pass through the micropillars (104 and 105) to reach the cells growing in the inner area (101) of the cell culture chamber (10). Therefore, in one example, the cell culture chamber (10) of the cell culture device as disclosed herein comprises 1 row to 5 rows of micropillars (104 and 105) separating the outer area (103) and inner area (101) of the cell culture chamber (10). In another example, the cell culture chamber (10) comprises 2 to 4 rows of micropillars (104 and 105). In another example, the cell culture chamber (10) comprises at least 1 row, at least 2 rows, at least 3 rows or at least 4 rows of micropillars (104 and 105). In another example, the cell culture chamber comprises 1 row, 2 rows, 3 rows, 4 rows or 5 rows of micropillars (104 and 105). In a preferred example, the cell culture chamber comprises 3 rows of micropillars (104 and 105) separating the outer area (103) and the inner area (101) of the cell culture chamber (10).

In another example, the micropillars (104) separating the inner area (101) and outer area (103) of the cell culture chamber (10) are arranged in a manner to prevent the content of the cell culture chamber from leaving (leaking out of) the inner area (101) and to allow cell culture medium to fill the outer area (103) to surround the inner area of the cell culture chamber (10). Therefore, in one example, the cell culture chamber comprises at least 2 rows, at least 3 rows or at least 4 rows of micropillars (104 and 105) and wherein the micropillars between the different rows are in a staggered arrangement (105). In another example, the cell culture chamber comprises at least 2 rows of micropillars (104) in a staggered arrangement (105). In another example, the cell culture chamber comprises at least 3 rows of micropillars (104) in a staggered arrangement (105). The staggered arrangement of micropillars (105) is illustrated in FIG. 1C (zoomed-in top view). It illustrates that the micropillars (105) in the different rows are alternated so that every opening formed by two micropillars in one row is faced directly by a micropillar in the following row.

The size of the micropillars (104) and the space between adjacent micropillars (104) placed in the cell culture chamber (10) of the cell culture device as disclosed herein serve to prevent leakage of the sample or cell mixture during the loading step and during subsequent growth of the cells in the inner area of the cell culture chamber, while allowing permeation of nutrients in the cell culture medium filling the outer area of the cell culture chamber into the cells cultured in the inner area of the cell culture chamber. If the space between adjacent micropillars (104) is too wide, the micropillars may not effectively prevent lateral movement of the cell mixture at the time of loading. This would result in formation of cell aggregates which would not be round, thus making subsequent analysis difficult. On the other hand, if the space between adjacent micropillars (104) is too narrow, this may hinder the exchange of nutrients between the inner area (101) and cell culture medium in the outer area (103) of the cell culture chamber (10), leading to poor cell conditions due to nutrient deficiency.

For the purpose of preventing leakage of the cell mixture from the inner area (101) of the cell culture chamber, the micropillars (104) can be in different shapes, for example, circular, triangular, rectangular, squarish, diamond-shaped or polygonal. In a preferred example, the micropillars (104) are rectangular in shape. Thus, in one example, the micropillars (104) have a cross-sectional dimension of about 50 μm to about 150 μm in length and about 25 μm to about 75 μm in width. In another example, the micropillars (104) have a dimension of about 75 μm to about 125 μm in length and about 40 μm to about 60 μm in width. In another example, the micropillars (104) have a dimension of about 80 μm to about 110 μm in length and about 45 μm to about 55 μm in width. In another example, the micropillars (104) have a dimension of about 50 μm, about 60 μm, about 70 μm, about 80 μm, about 90 μm, about 100 μm, about 110 μm, about 120 μm, about 130 μm, about 140 μm or about 150 μm in length and about 25 μm, about 30 μm, about 35 μm, about 40 μm, about 45 μm, about 50 μm, about 55 μm, about 60 μm, about 65 μm, about 70 μm or about 75 μm in width. In a preferred example the micropillars (104) have a dimension of about 100 μm in length and about 50 μm.

In another example, adjacent micropillars in the at least one row of micropillars are spaced apart from each other at about 50 μm to about 500 μm. In another example, adjacent micropillars in the at least one row of micropillars (104) are spaced apart from each other at about 100 μm to about 400 μm, about 150 μm to about 350 μm, about 200 μm to about 300 μm or about 75 μm to about 125 μm. In another example, adjacent micropillars (in the at least one row of micropillars (104) are spaced apart from each other at about 90 μm to about 110 μm. In yet another example, adjacent micropillars in the at least one row of micropillars (104) are spaced apart from each other at about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 275,μm about 300 μm, about 325 μm, about 350 μm, about 375 μm about 400 μm, about 425 μm about 450 μm, about 475 μm or about 500 μm. In one example, the space between adjacent micropillars is about 100 μm. In another example, micropillars of the at least one row of micropillars (104) separating the outer area (103) and inner area (101) of the cell culture chamber (10) have a dimension of about 100 μmin length and about 50 μm and are spaced about 100 μm apart. In a preferred example, the outer area (103) and the inner area (101) of the cell culture chamber are separated by 3 rows of micropillars (104 and 105) in a staggered arrangement, wherein the size of each micropillar (104) is about 100 μm in length and about 50 μm in width and wherein the space between adjacent micropillars is about 100 μm.

In another example, the cell culture chamber (10) of the cell culture device as disclosed herein comprises at least two rows of micropillars (104) separating the inner area (101) and the outer area (103) of the cell culture chamber, and wherein the micropillars (104) between the different rows are in a staggered arrangement. In another example, the space between each adjacent rows of micropillars in the at least two rows of micropillars is about 50 μm to about 500 μm. In some examples, the space between each adjacent rows of micropillars in the at least two rows of micropillars is about 100 μm to about 400 μm, about 150 μm to about 350 μm, about 200 μm to about 300 μm or about 75 μm to about 125 μm. In yet another example, the space between each adjacent rows of micropillars in the at least two rows of micropillars is about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 275, μm about 300 μm, about 325 μm, about 350 μm, about 375 μm about 400 μm, about 425 μm about 450 μm, about 475 μm or about 500 μm.

In a specific example as shown in FIG. 1C, the cell culture chamber (10) of the cell culture device as disclosed herein is circular, has a dimension of 6 mm in diameter and 300 μm in depth, comprises an inlet (102) having a 1 mm diameter created in the centre of the cell culture chamber for the loading of sample or a cell mixture, wherein the inner area (101) and outer area (103) of the cell culture chamber are separated by three concentric rows of micropillars (104 and 105), wherein each of the micropillars measures about 100 μm in length, about 50 μm in width and about 300 μm in height, and wherein the space between adjacent micropillars in the same row and the space between adjacent rows of micropillars is about 100 μm.

In another example, micropillars (107) are also found inside the inner area (101) of the cell culture chamber (10). The micropillars (107) inside the inner area (101) of the cell culture chamber (10) are sparsely arranged. As used herein, the term “sparsely arranged” refers to the arrangement of the micropillars (107) in a dispersed manner and in small numbers. The sparsely arranged micropillars (107) inside the inner area (101) of the cell culture chamber (10) are designed to slow down and evenly distribute the flow of the cells or the cell mixture during the loading process. For example, when the cell mixture is loaded through the inlet (102) and enters the inner area (101) of the cell culture chamber (10), the cells in the mixture diffuse freely in all directions. The sparsely arranged micropillars can slightly slow down the faster-flowing liquid, allowing the cell mixture to reach the edge of the inner area enclosed by the micropillars (104), at approximately the same time and uniformly fill the entire inner area (101). This results in the cell aggregates forming a circular outer shape. Accordingly, the number of micropillars (107) inside the inner area (101) of the cell culture chamber (10) is less than the number of micropillars (104) separating the inner area (101) and outer area (103) of the cell culture chamber. In addition, the space between adjacent micropillars (107) in the inner area (101) of the cell culture chamber is wider than the space between adjacent micropillars (104) separating the inner area (101) and the outer area (103) of the cell culture chamber (10).

The micropillars (107) inside the inner area (101) of the cell culture chamber (10) also provide structural support for the inner area (101) of the cell culture chamber (10). The micropillars (107) may be arranged in spiral pattern with respect to the cell loading inlet (102) or in a circular pattern around the inlet (102) of the inner area (101) of the cell culture device (10). In one example, the inner area (101) comprises at least 1 row of micropillars inside the inner area (101) arranged in a circular pattern around the inlet (102). In other examples, the inner area (101) comprises at least 2 rows, at least 3 rows, or at least 4 rows of micropillars (107) inside the inner area (101) of the cell culture chamber (10). In some examples, the inner area (101) comprises 1 row, 2 rows, 3 rows, or 4 rows of micropillars (107) inside the inner area (101) of the cell culture chamber (10). In a preferred example, the inner area comprises 2 rows of micropillars (107) inside the inner area (101) of the cell culture chamber (10) arranged in a circular pattern around the cell loading inlet (102).

During the cell loading process, the cells or a mixture comprising cells and a scaffold is introduced into the inner area (101) of the cell culture chamber (10). When the cell mixture is loaded into the inner area (101), the presence of micropillars (107) inside the inner area (101) of the cell culture chamber (10) slows down the flow of the cell mixture and thereby allowing an even distribution of the cell mixture in the inner area (101) of the cell culture chamber (10). Therefore, the space between adjacent micropillars (107) inside the inner area (101) of the cell culture device should be wide enough to allow the flow of the cell mixture. In one example, adjacent micropillars (107) of the at least one row of micropillars (107) inside the inner area (101) of the cell culture chamber (10) are spaced apart from each other at about 400 μm to about 1200 μm. In another example, the micropillars (107) are spaced about 500 μm to about 1000 μm or about 600 μm to about 800 μm. In some examples, adjacent micropillars (107) of the at least one row of micropillars (107) inside the inner area (101) of the cell culture chamber (10) are spaced apart from each other at about 400 μm, about 500 μm, about 600 μm, about 700 μm, about 800 μm, about 900 μm, about 1000 μm, about 1100 μm or about 1200 μm.

In another example, the space between adjacent micropillars (107) inside the inner area of the cell culture chamber is wider than the space between adjacent micropillars (104) separating the inner area (101) and outer area (103) of the cell culture chamber (10). Since the function of the micropillars (107) inside the inner area (101) of the cell culture chamber (10) serves to slow down flow of the cells or the cell mixture to allow an even distribution of the cells in the inner area (101) of the cell culture chamber (10), the space between adjacent micropillars (107) is wider than the space between adjacent micropillars (104) separating the inner area (101) and outer area (103) of the cell culture chamber (10), which serve to prevent leakage of the cells or cell mixture out of the inner area (101) and to facilitate permeation of nutrients from the cell culture medium to the cells or cell mixture. In another example, the space between adjacent micropillars (107) inside the inner area of the cell culture chamber is about 2 times to about 10 times, about 4 times to about 8 times, about 5 times to about 6 times, about 2 times to 6 times, about 3 times to 5 times or about 4 times to about 5 times wider than the space between adjacent micropillars (104) separating the inner area (101) and outer area (103) of the cell culture chamber (10). In some examples, the space between adjacent micropillars (107) inside the inner area of the cell culture chamber is about 2 times, about 3 times, about 4 times, about 5 times, about 6 times, about 7 times or about 8 times wider than the space between adjacent micropillars (104) separating the inner area (101) and outer area (103) of the cell culture chamber (10).

In another example, the number of micropillars (107) inside the inner area of the cell culture chamber is less than the number of micropillars (104) separating the inner area (101) and outer area (103) of the cell culture chamber (10). In some examples, the number of micropillars (107) inside the inner area of the cell culture chamber about 400% to about 90% less, about 50% to about 80% less, about 50% to about 75% less, about 50% to about 60% less, about 60% to about 90% less, about 60% to about 80% less, about 60% to about 75% less, about 70% to about 90% less, about 70% to 85% less, about 75% to about 85% less, about 80% to about 90% less or about 85% to 90% less than the number of micropillars (104) separating the inner area (101) and outer area (103) of the cell culture chamber (10). In some examples, the number of micropillars (107) inside the inner area of the cell culture chamber about 40% less, about 50% less, about 60% less, about 70% less, about 75% less, about 80% less, about 85% less, about 90% less than the number of micropillars (104) separating the inner area (101) and outer area (103) of the cell culture chamber (10).

In another example, the inner area (101) of the cell culture chamber (10) comprises at least 2 rows of micropillars (107). In another example, the space between each adjacent rows of micropillars in the at least one row of micropillars is about 400 μm to about 1200 μm.

The dimensions of the micropillars (107) inside the inner area (101) of the cell culture chamber (10) may be different or identical to the micropillars (104) separating the inner area (101) and outer area (103) of the cell culture chamber (10). Thus, the micropillars (107) can be in different shapes, for example, circular, triangular, rectangular, squarish, diamond-shaped or polygonal. In a preferred example, the micropillars (107) are rectangular in shape. In one example, the micropillars (107) have a cross-sectional dimension of about 50 μm to about 150 μm in length and about 25 μm to about 75 μm in width. In another example, the micropillars have a dimension of about 75 μm to about 125 μm in length and about 40 μm to about 60 μm in width. In another example, the micropillars have a dimension of about 80 μm to about 110 μm in length and about 45 μm to about 55 μm in width. In another example, the micropillars have a dimension of about 50 μm, about 60 μm, about 70 μm, about 80 μm, about 90 μm, about 100 μm, about 110 μm, about 120 μm, about 130 μm, about 140 μm or about 150 μmin length and about 25 μm, about 30 μm, about 35 μm, about 40 μm, about 45 μm, about 50 μm, about 55 μm, about 60 μm, about 65 μm, about 70 μm or about 75 μm in width. In a preferred example the micropillars have a dimension of about 100 μm in length and about 50 μm.

In one example, the height of the micropillars (107) is about 100 μm to about 500 μm. In another example, the height of the micropillars (107) is about 200 μm to about 400 μm or about 250 μm to about 350 μm. In another example, the height of the micropillars (107) is about 270 μm to about 330 μm. In another example, the height of the micropillars (107) is about 280 μm to about 320 μm. In another example, the height of the micropillars (107) is about 100 μm, about 150 μm, about 200μm, about 250 μm, about 260 μm, about 270 μm, about 280 μm, about 290 μm, about 300 μm, about 310 μm, about 320 μm, about 330 μm, about 340 μm, or about 350 μm, about 360 μm, about 370 μm, about 380, about 390 μm, about 400 μm, about 450 μm or about 500 μm. In another example, the height of the micropillars (107) is about 300 μm.

In another example, the cell culture device as disclosed herein comprises one or more cell culture medium reservoirs (11) that are fluidically connected to the cell culture chamber (10), so that when one of the cell culture medium reservoirs (11) is filled with a cell culture medium, the cell culture medium can flow into the outer area (103) of the cell culture chamber to surround the inner area (101) of the cell culture chamber and into the other cell culture medium reservoirs (11). For example, the cell culture medium can flow from the cell culture medium reservoir to the outer area of the cell culture chamber to surround the cells in cultured in the inner chamber so as to allow permeation of nutrients from the peripheral to the inner area via diffusion. This design eliminates the need for an external flow system for supplying cell culture medium to the cell culture chamber.

Therefore, in one example, the cell culture device comprises at least 1 cell culture medium reservoir (11). In another example, the cell culture device comprises at least 2 cell culture medium reservoirs (11). In another example, the cell culture device comprises 1 to 6 cell culture reservoirs (11). In another example, the cell culture device comprises 2 to 4 cell culture reservoirs (11). In another example, the cell culture device comprises 1, 2, 3, 4, 5 or 6 cell culture reservoirs (11). In another example, the cell culture device comprises 2 cell culture reservoirs (11). In a preferred example, the cell culture device comprises 2 cell culture medium reservoirs (11) arranged on opposite ends of the cell culture chamber (10) and are fluidically connected to the outer area (103) of the cell culture chamber.

The one or more cell culture medium reservoirs (11) can be of any shape, such as rectangular, squarish or circular. In one example, the cell culture medium reservoirs are circular. Thus, in one example, the diameter of the cell culture medium reservoirs (11) is about 8 mm to about 18 mm. In another example, the diameter of the cell culture medium reservoirs (11) is about 10 mm to about 16 mm. In another example, the diameter of the cell culture medium reservoirs (11) is about 11 mm to about 15 mm. In another example, the diameter of the cell culture medium reservoirs (11) is about 12 mm to about 13 mm. In some examples, the diameter of the cell culture medium reservoirs (11) is about 8 mm, about 9 mm, about 10 mm, about 11 mm, about 12 mm, about 13 mm, about 14 mm, about 15 mm, about 16 mm, about 17 mm, or about 18 mm. In a preferred example as shown in FIG. 1C-1E , the diameter of the cell culture medium reservoirs (11) is 12 mm.

The top component (1) of the cell culture device as shown for example in FIGS. 1C and 1D is manufactured from a material suitable for culturing cells, particularly tumor cells, so that a tumor microenvironment can be established on the cells cultured in the cell culture device. In one example, the top component (1) of the cell culture device is fabricated from a material selected from the group consisting of glass, silicon and a polymer. In another example, the top component of the cell culture device is fabricated from a polymer. In another example, the top component of the cell culture device is fabricated from a polymer, selected from a group consisting of polydimethylsiloxane (PDMS), polymethylmethacrylate (PMMA), polycarbonate (PC) or cyclic olefin copolymer (COC). In another example, the top component of the cell culture device is fabricated from polydimethylsiloxane (PDMS). As can be understood by a person skilled in the art, the material for fabricating the top component (1) of the cell culture device should be transparent to facilitate imaging and photography of the cells cultured in the cell culture device as disclosed herein.

The cell culture device as disclosed herein comprises a top component (1) bonded to a bottom substrate (2). A person skilled in the art will be aware of methods of bonding one material to the other, such as through using uncured form of the material used to fabricate the top component of the cell culture device. For example, uncured polydimethylsiloxane (PDMS) can be used to attach the top component (1) to the bottom substrate (2) so that after the molten PDMS solidifies, the top component (1) is bonded to the bottom substrate (2). The top component (1) comprises a cell culture chamber (10) comprising an outer area (103) and an inner area (101) separated by at least one row of micropillars (104 and 105), and the at least one cell culture reservoirs (11) fluidically connected to the outer area (103) of the cell culture chamber. As used herein, the term “substrate” refers to a surface material on which cells are cultured on. In one example, the bottom substrate (2) is made using a material selected from the group consisting of glass, polystyrene and polycarbonate. In another example, the bottom substrate (2) is made using glass. The substrate can be made of the same or different material as the top component as described herein. The material of the bottom substrate (2) is transparent to facilitate imaging and monitoring of the cell which are cultured in the cell culture device with conventional inverted fluorescence microscopy.

The cell culture device as disclosed herein has dimensions which allow placement of the cell culture device inside a cell culture dish or the well of a multi-well cell culture plate. In one example, the cell culture dish is a 6-cm dish. In another example, the cell culture plate is a 10-cm dish. In another example, the cell culture plate is a multi-well cell culture plate. In another example, the cell culture plate is a six-well cell culture plate or a 12-well cell culture plate. In another example, the cell culture plate is a six-well cell culture plate. The ability to fit the cell culture device into the well of the standard six-well plate allows use of the cell culture device as disclosed herein with common microscope platforms, thereby facilitating batch operations and real-time monitoring.

In a preferred example, the cell culture device as disclosed herein is designed to fit into the well of the standard six-well cell culture plate. As known in the art, the internal diameter of a well in the six-well plate is about 35 mm. Thus, in one example the cell culture device comprises a top component (1) bonded to a bottom glass substrate (2) having a diameter of less than about 35 mm. In another example, the cell culture device is about 34 mm, about 33 mm, about 32 mm, about 31 mm, about 30 mm, about 28 mm, about 26 mm, about 24 mm or about 20 mm. In a preferred example. The cell culture device is about 34 mm.

In another aspect, the present disclosure relates to a method of manufacturing the cell culture device as disclosed herein, comprising the steps of a) creating a mold of the top component (1) of the cell culture device as disclosed herein, b) pouring a polymer in liquid form over the mould, c) detaching the polymer from the mould after the polymer becomes solidified, d) bonding the polymer with a bottom substrate (2) and, e) obtaining the cell culture device.

In another example, the polymer used in the step for creating a mould of the top component (1) of the cell culture device as disclosed herein is selected from the list consisting of polydimethylsiloxane (PDMS), polymethylmethacrylate (PMMA), polycarbonate (PC) or Cyclic olefin copolymer (COC). In a preferred example, the polymer is polydimethylsiloxane (PDMS).

In another example, the mold of the top component (1) of the cell culture device is created using photolithographic procedures known in the art.

In another example, the bottom substrate (2) that is bonded to the polymer corresponding to the top component (1) of the cell culture device, is made of a material selected from the list consisting of glass, polystyrene and polycarbonate. In a preferred example, the bottom substrate (2) is made of glass.

In another aspect, the present disclosure relates to a method of culturing cells using the cell culture device as disclosed herein. In one example, the method for culturing cells comprises the steps of: a) loading a mixture comprising cells and a scaffold into the inner area (101) of the cell culture chamber (10) of the cell culture device as disclosed herein through the inlet (102) until the mixture covers the inner area (101) of the cell culture chamber enclosed by the micropillars (104 and 105); b) adding a cell culture medium to the one cell culture reservoirs (11); c) sealing the inlet (102) of the cell culture chamber loaded with the mixture with a cover; and d) culturing the cells to obtain a cell aggregate in the inner area (101) of the cell culture chamber (10). The steps of the method for culturing cells using the cell culture device as disclosed herein are described in FIG. 1F. In another example, the sealing of the inlet can be performed before the step of adding a cell culture medium to the cell culture reservoir.

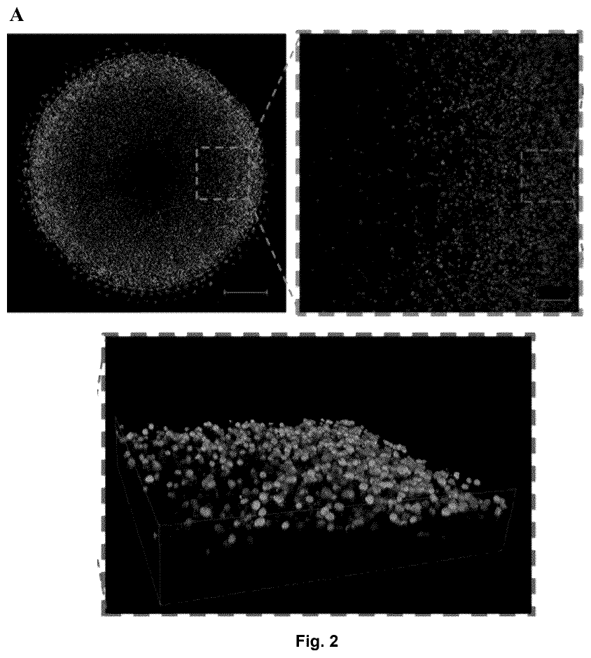

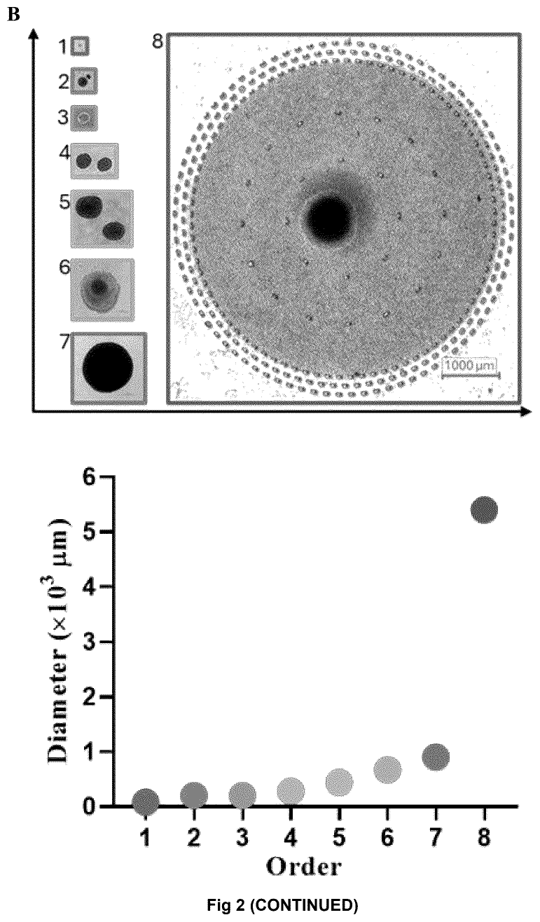

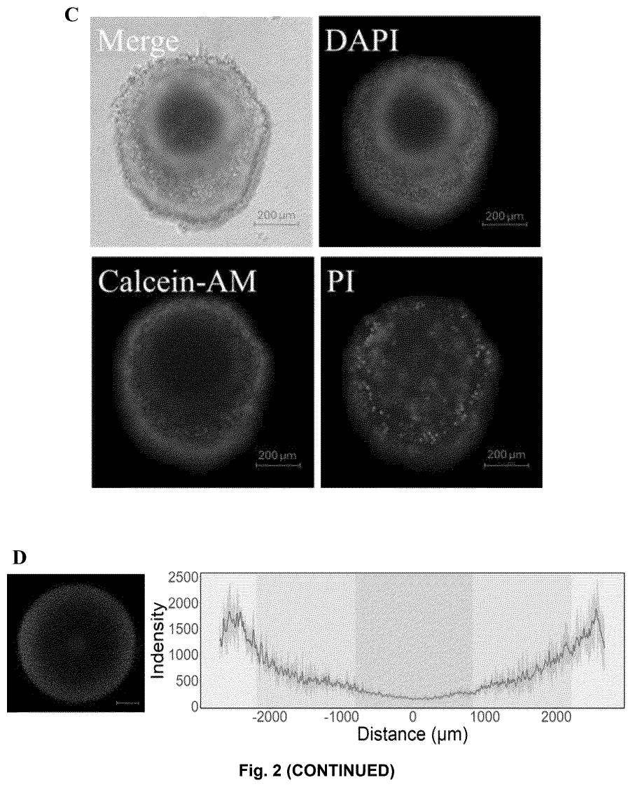

In one example, the method of culturing cells as disclosed herein comprises culturing of tumor cells in the cell culture device as disclosed herein. In another example, the tumor cells are SK-Hep-1 cells. In some examples, the cells are modified to express green fluorescent protein (GFP) or pre-incubated with a fluorescent probe to facilitate real-time monitoring of the cells. In one example, the fluorescent probe is selected from the group consisting of DAPI, pHrodo Red AM, BCECF AM and Green Hypoxia Reagent, or a combination thereof. As described in FIGS. 2, 3 and 4, culturing of SK-Hep-1 tumor cells in the cell culture device as disclosed herein using method as disclosed herein replicates the conditions required for the formation of a tumor microenvironment comprising a gradient as the design of the cell culture device as disclosed herein allows nutrients and oxygen to diffuse exclusively from the periphery to the inner core.

As used herein, the term “tumor microenvironment” refers to a complex ecosystem surrounding a tumor. As known in the art, the tumor microenvironment is composed of cellular constituents that include cells of the vasculature, infiltrating immune cells, and resident fibroblasts, adipocytes and nerves, and a noncellular component composed of diverse soluble factors and a highly variable and evolving insoluble extracellular matrix (ECM). One key feature of the tumor microenvironment is the existence of local chemical and physical gradients. Thus, as used herein, the term “gradient microenvironment” refers to existence of chemical and physical gradient within a tumor, a tumor spheroid, an aggregate of cells or cell aggregate obtained from the method of culturing cells as disclosed herein. Examples of gradients in the tumor microenvironment include, but are not limited to pH gradient, stiffness gradient, oxygen gradient, growth factor and/or chemokine concentration gradient, and electrical gradient.

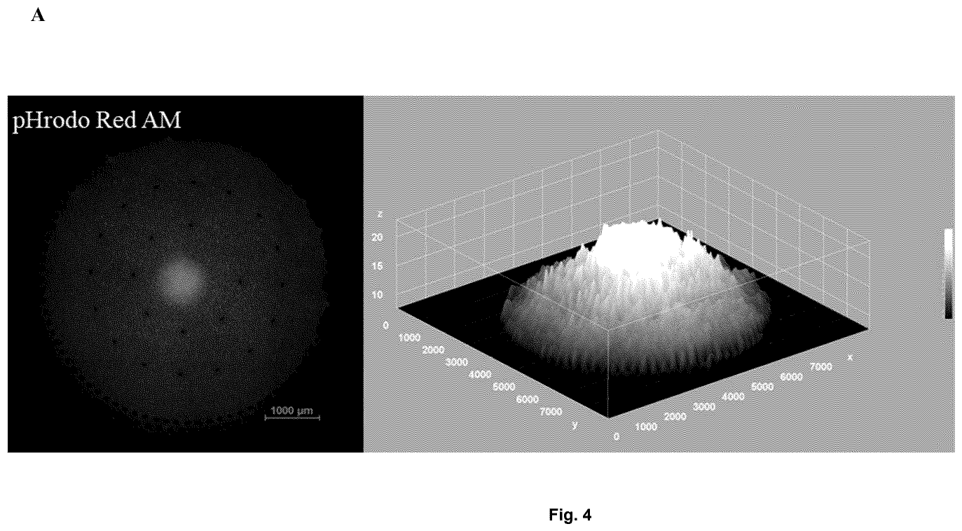

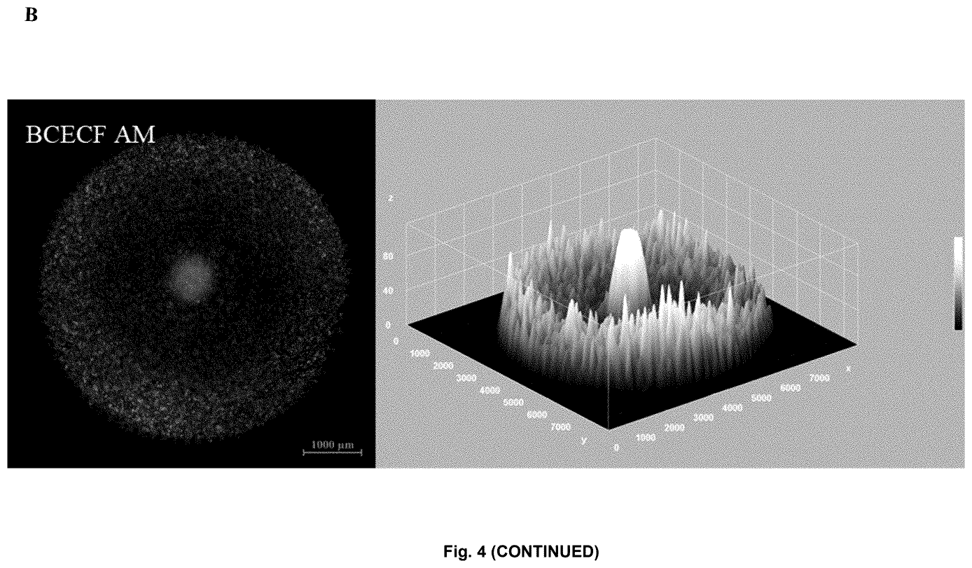

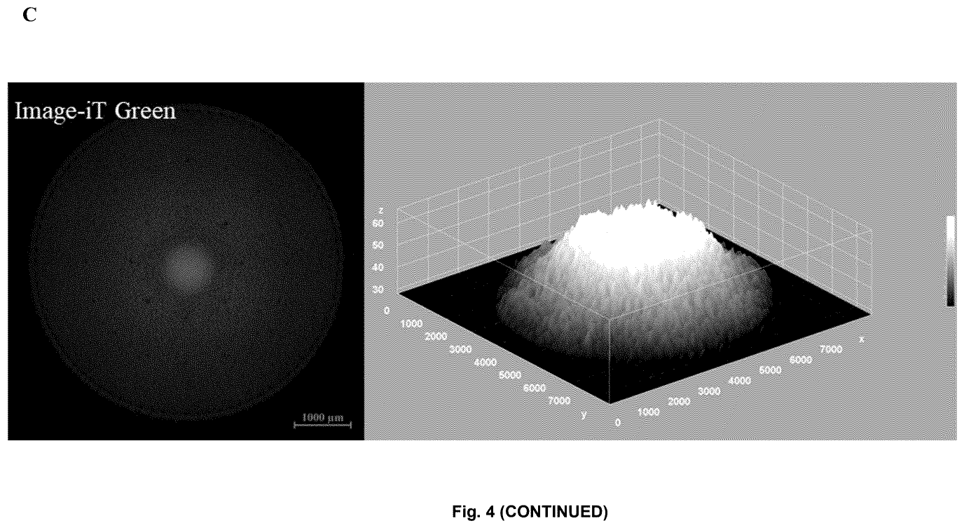

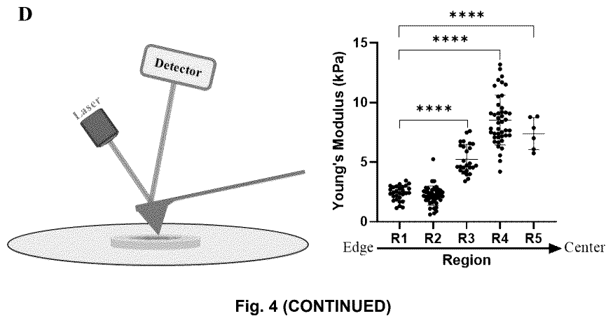

As described herein, the method of culturing tumor cells in the cell culture device as disclosed herein recapitulates the gradient distribution of cell proliferation status (FIG. 2), pH and oxygen levels (FIGS. 4A, B and C) in the tumor microenvironment when culturing cells using the cell culture device as disclosed herein.

The stiffness feature can also be established when culturing tumor cells in the cell culture device as disclosed herein. Current research on tumor spheroid stiffness mainly focuses on the overall stiffness profile of the tumor spheroid, as the characterization of stiffness distribution features within a tumor spheroid is challenging due to the restrictions imposed by the tumor spheroid enclosed spherical structure. Thus, one feature of the method of culturing cells in the cell culutre device as disclosed here is that the projection of a 3D tumor spheroid into a 2D plane allows for direct measurement of stiffness in different areas across the microenvironment gradient.

Therefore, by projecting the 3D tumor spheroid features onto a 2D thin glass plane based on the cell culture device and the method of culturing cells as disclosed herein, the 2D-plane observation of cellular profiles within the tumor spheroids'internal region is facilitated.

In another example, the method for culturing cells as disclosed herein comprises culturing of a mixture comprising cells and a scaffold, in the cell culture device as disclosed herein. The term “cell mixture” as used herein also refers to the mixture comprising cells and a scaffold.

In a further example, the cell mixture cultured in the cell culture device as disclosed herein comprises one population of cells. In another example, the cell mixture cultured in the cell culture device as disclosed herein comprises multiple population of cells. In another examples, the cell mixture cultured in the cell culture device as disclosed herein comprises tumor cells, stromal cells, cancer-associated fibroblasts, immune cells, or a combination thereof.

As disclosed herein, cells for culturing in the method as disclosed herein are mixed with a scaffold. Common scaffolds known in the art include, but are not limited to alginate, collagen, gelatin, gelatin methacrylate, hydrogels and polycaprolactone. Thus, in one example, the method for culturing cells as disclosed herein comprises culturing of cells mixed with a scaffold in the cell culture device as disclosed herein, wherein the scaffold is selected from the group consisting of alginate, collagen, gelatin, gelatin methacrylate, hydrogels and polycaprolactone or a combination thereof. In one example, scaffold formation within the inner area (101) of the cell culture device can take place via complex coacervation which takes place between the different polymers, e.g. collagen and a terpolymer, comprising the cells. In another example, the method for culturing of cells in the cell culture device as disclosed herein comprises culturing of tumor cells mixed with gelatine methacrylate. In a further example, the gelatin methacrylate is present in the cell mixture at about 5% to about 15%. In some examples, the gelatin methacrylate is present in the cell mixture at about 7% to about 13%. In other examples, gelatin methacrylate is present in the cell mixture at about 9% to about 11%. In another example, gelatin methacrylate is present in the cell mixture at about 5%, 6%, 7%, 8%, 9%, 10%, 11%, 12%, 13%, 14% or 15%. In a preferred example, the cells are mixed with 10% of gelatin methacrylate and loaded in the inlet (102) of the cell culture device as disclosed herein. In another example, the cells are enzymatically dissociated and centrifuged to acquire cell pellets before combining with 10% gelatin methacrylate to form the mixture comprising cells and a scaffold as disclosed herein.

The mixture comprising cells and a scaffold as described herein can be prepared for loading into the inlet (102) by dissociating the cells using common enzymes, including but not limited to trypsin and collagenase. As can be appreciated by a person skilled in the art, other non-enzymatic methods of cell dissociation can also be applied to prepare the cells to obtain a cell pellet for mixing with a suitable scaffold.

In another example, the method of culturing cells as disclosed herein comprises loading of the mixture comprising cells and a scaffold into the cell culture chamber of the cell culture device as disclosed herein through the inlet (102), until the mixture completely covers the inner area (101) of the cell culture chamber enclosed by the micropillars (104 and 105). In a further example, the mixture comprising tumor cells and 10% gelatin methacrylate are solidified by applying a brief exposure of UV light after the cells were loaded into the cell culture chamber of the device as disclosed herein. In another example, the mixture comprising tumor cells and 10% gelatin methacrylate is exposed to the UV light for about 10 s to about 15 s at a power of about 60 mW cm⁻² and at a wavelength of about 405 nm. Once the mixture comprising tumor cells and 10% gelatin methacrylate are solidified, the inlet (102) of the cell culture device as disclosed herein is covered with a thin cover. In one example, the cover is a glass cover. The purpose of the cover is to seal the cell loading inlet (102) and to prevent diffusion of oxygen to the cells in the cell culture chamber from the top surface.

After the mixture comprising cells and a scaffold, wherein the cells, for example, are tumor cells and the scaffold is 10% gelatin methacrylate, has been solidified, a cell culture medium is added into the one of more cell culture reservoirs (11) of the cell culture device and the opening of the inlet (102) on the top surface of the cell culture device is sealed with a small cover. As a person skilled in the art can appreciate, the cell culture medium for culturing cells is selected based on the type of cells to be cultured. After filling the cell culture medium reservoirs (11) with the appropriate medium, the cell culture device is placed inside the well of a cell culture plate and kept in an incubator under standard cell culture conditions. In one example, the cell culture plate is a multi-well cell culture plate. In another example, the cell culture plate is a six-well cell culture plate. During the culturing process, the cell culture medium in the cell culture reservoir (11) is replaced regularly to maintain optimal growth conditions of the cells. As is known in the art, the interval of replacing cell culture medium is dependent on the type of cells that is being cultured. In one example, the cell culture medium in the reservoirs (11) is replaced every day. In another example, the cell culture medium in the cell culture reservoirs (11) is replaced every two days.

The mechanism of gradient formation in a tumor spheroid primarily relies on the natural penetration gradient of nutrients from the periphery to the inner area, in conjunction with other cellular metabolic processes. Therefore, the density of cells in the mixture comprising cell and a scaffold that is initially loaded into inner area of the cell culture device for culturing in the cell culture device as disclosed herein determines the final gradient microenvironment. In one example, the starting density of cells loaded into the cell culture device is about 3.125×10⁴ cells to about 1×10⁶ cells in about 10 μL of scaffold. In another example, the starting density of cells loaded into the cell culture device is about 1.25×10⁵ cells to about 5×10⁵ in about 10 μL of scaffold. In another example, the starting density of cells loaded into the cell culture device is about 5×10⁵ cells to about 1×10⁶ cells in about 10 μL of scaffold. In another example, the starting density of cells loaded into the cell culture device is about 1.25×10⁵ cells, about 2.5×10⁵ cells, about 5×10⁵ cells or about 1×10⁶ cells in about 10 μL of scaffold. In a further example, the scaffold is gelatin methacrylate. In another example, the gelatin methacrylate is present at a concentration of 10% in the mixture.

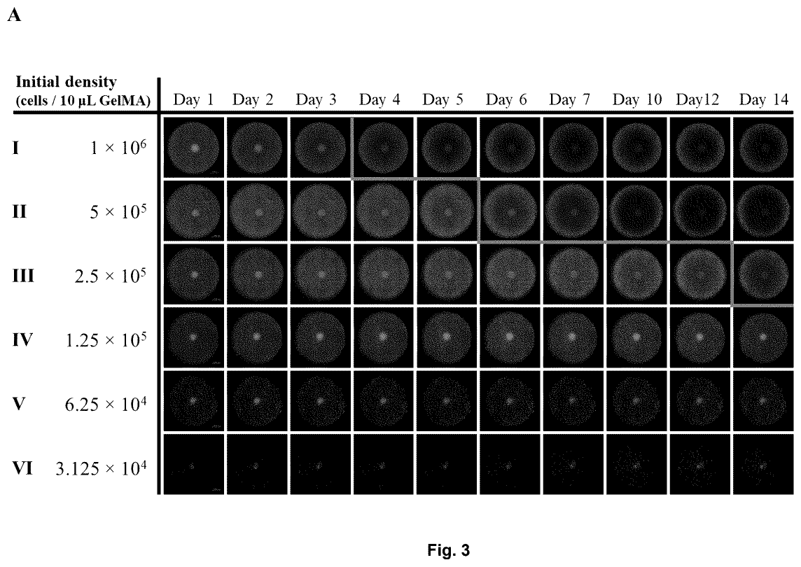

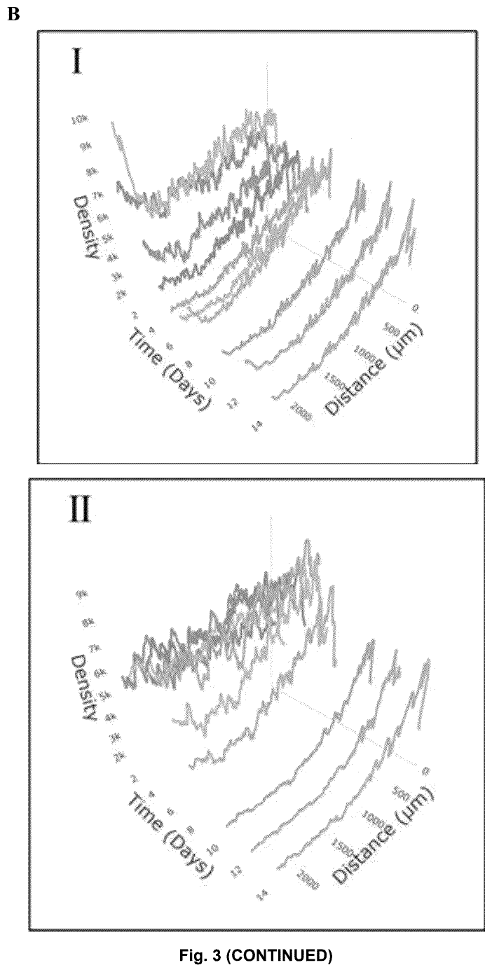

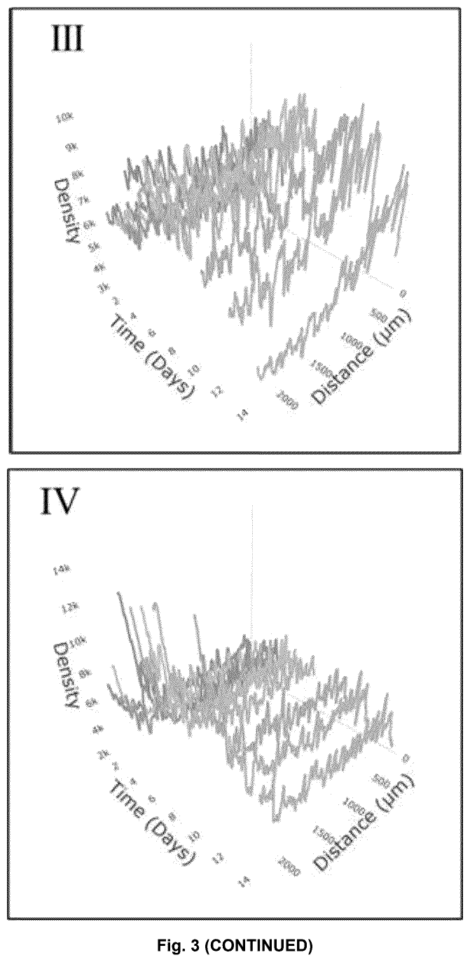

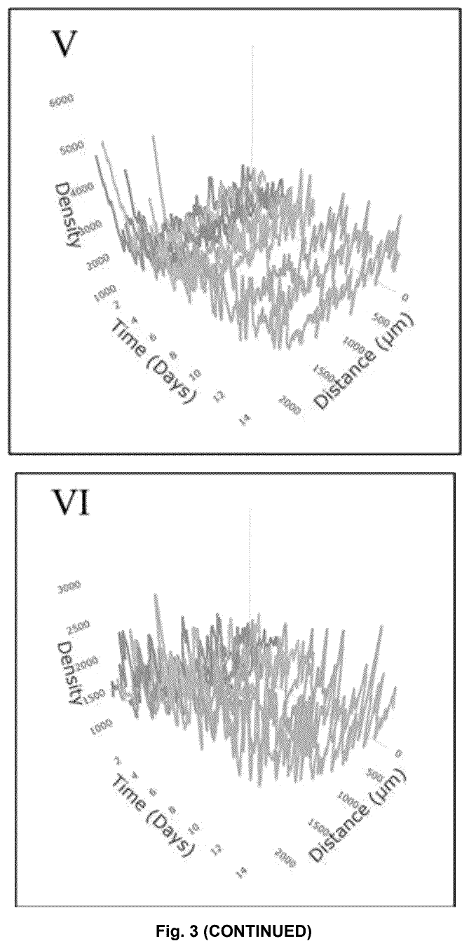

As described herein in FIG. 3A, a starting density of about 1×10⁶ cells per 10 μL of 10% gelatin methacrylate resulted in the obvious appearance of tumor microenvironment gradient characteristics from Day 4 of culturing in the cell culture device as disclosed herein. However, when the density of cells loaded is reduced to about 5×10⁵ cells per 10μL of 10% gelatin methacrylate and 2.5×10⁵ cells per 10 μL of 10% gelatin methacrylate, the gradient features only began to appear from day 6 and day 14, respectively.

Therefore, a tumor gradient microenvironment can be established by culturing cells in the cell culture device as disclosed herein for about 4 days to about 14 days. In one example, the method of culturing cells as disclosed herein comprises culturing of cells in the cell culture device for about 4 days to about 14 days. In some examples, the cells are cultured in the device as disclosed herein for about 6 days to about 12 days. In another example, the cells are cultured in the device as disclosed herein for about 8 days to 10 days. In yet another example, the cells are cultured in the cell culture device for about 4 days, about 5 days, about 6 days, about 7 days, about 8 days, about 9 days, about 10 days, about 11 days, about 12 days, about 13 days, or about 14 days. In a preferred example, to establish a tumor microenvironment, 1×10⁶ cells per 10 μL of 10% gelatin methacrylate are cultured in the cell culture device as disclosed herein for 4 days. In a further example, 5×10⁵ cells per 10 μL of 10% gelatin methacrylate are cultured in the cell culture device as disclosed herein for 6 days. In another example, 2.5×10⁵ cells per 10 μL of 10% gelatin methacrylate are cultured in the cell culture device as disclosed herein for 14 days.

Cell aggregates obtained from cells cultured in the cell culture device as disclosed herein with a starting cell density lower than 2.5×10⁵ cells per 10 μL of 10% gelatin methacrylate only formed the gradient microenvironment slowly within the monitored 14 days. The trend in fluorescence intensity over time shows that as starting density of cells loaded into the device decreases, nutrient consumption rate gradually falls below diffusion rate, making it difficult to establish the gradient environment (FIG. 3B). It should be noted that the starting density of cells cannot be infinitely increased, as 1×10⁶ cells in the cell pellet occupy nearly a volume of 10 μL, hence, further increasing the density cell of cells in the cells and gelatin methacrylate mixture would make the mixture too viscous for loading. The cells remaining in the channel (106) of the cell loading inlet gradually died as the gradient environment was established and has minimal impact on the final microenvironment phenotype.

When culturing cells in the cell culture device using the method as disclosed herein, the mixture of tumor cells and gelatin methacrylate will integrate in the cell culture device and is referred to as a “cell aggregate”. As used herein, the term “cell aggregate” refers to the product obtained from culturing a cell mixture using the cell culture device as disclosed herein. During the culturing process, the initial cell mixture develops into the cell aggregate to form a tumor gradient microenvironment resembling that of a 3D tumor spheroid as described in FIG. 2A. The cell aggregate thus comprises a proliferative outer layer, a quiescent middle layer and a necrotic inner core. In one example, the cell aggregate comprises a gradient distribution characteristic in pH, oxygen availability or stiffness. In another example, the pH of the cell aggregate decreases from the outer proliferative layer to the inner necrotic core. In a further example, the oxygen concentration of the cell aggregate decreases from the outer proliferative layer to the inner necrotic core. In yet another example, the stiffness of the cell aggregate obtained from the method of culturing cells as disclosed herein increases from the outer proliferative layer to the inner necrotic core.

The cell aggregate obtained from the method of culturing cell as disclosed herein can be removed from the cell culture device as disclosed herein with ease, thus allowing isolation and retrieval of cells from the different gradient regions for downstream histological, cellular and molecular analyses. Therefore, the method of culturing cells using the cell culture device as disclosed herein provides a tool for investigating the various gradient microenvironments in a tumor spheriod, including but not limited to chemotaxis effects, cell-cell interaction, drug resistance mechanisms, and immune infiltration. In one example, the cell aggregate obtained from the method of culturing cell as disclosed herein can be removed from the cell culture device as disclosed herein by dissembling the top component (1) comprising the cell culture chamber (10) and cell culture medium reservoirs (11) from the bottom substrate (2). When the top component (1) is separated from the bottom substrate (2), the cell aggregate will be attached to the bottom substrate (2). The cell aggregate on the bottom substrate (2) may be removed from the bottom substrate for downstream analysis. In one example, the cell aggregate can be separated into an outer, middle and inner region corresponding to an outer proliferative layer, an intermediate quiescent layer and an inner necrotic core of a tumor spheroid, for downstream histological, cellular and molecular analysis. In another example, the different gradient regions can be obtained from the cell aggregate using punches of different sizes to separate the different regions.

The size of the cell aggregate obtained from the cell culturing method as disclosed herein can be modified by modifying the size of the inner area (101) of the cell culture chamber (10). As described herein, the diameter of the inner area of the cell culture chamber is determined by the boundary set by the micropillars (104) separating the inner area and outer area of the cell culture device. For example, the diameter of the inner area (101) of the cell culture chamber of the device ranges from about 4 mm to about 10 mm. Therefore, in one example, the diameter of the cell aggregate obtained from the cell culturing method as disclosed herein is about 4 mm to about 10 mm. In another example, the diameter of the cell aggregate is about 6 mm to about 8 mm. In another example, the diameter of the cell aggregate is about 4 mm, 5 mm, 6 mm, 7 mm, 8 mm, 9 mm or 10 mm. In preferred example, the size of the cell aggregate obtained from the cell culturing method as disclosed herein is about 6 mm. As can be appreciated by a person skilled in the art, the volume of the cell aggregate obtained from culturing cells in the cell culture device is determined by the volume of the inner area (101) of the cell culture chamber.

Tumor spheroids obtained from traditional techniques such as suspension culture typically have diameters ranging from a few hundred micrometres. The size of these tumor spheroid makes standard direct immunofluorescence staining challenging due to reduced antibody penetration to the inner region of the tumor spheroid. Additionally, the smaller tumor spheroid obtained from traditional techniques also reduces the success rate of obtaining usable histological sections. Thus, the advantage of using the method for culturing cells in the cell culture device as disclosed herein is that the resultant cell aggregate obtained from the cell culturing method as disclosed herein has a diameter of about 6 mm in a preferred example, which provides a sizable sample for subsequent histological sectioning and cell extraction analysis.

The method of culturing cells using the cell culture device as disclosed herein replicates the 3D tumor spheroid-like gradient microenvironment on a 2D plane, which captures the classical proliferation phenotype of the tumor spheroid, comprising a peripheral proliferative layer, an intermediate quiescent layer and an inner neurotic core, together with key gradient features, including pH, hypoxia, and stiffness on the resultant cell aggregate obtained. Thus, the culturing of a cell mixture using the cell culture device as disclosed herein enables real-time observation and monitoring of gradient formation using standard imaging techniques. Furthermore, the resultant cell aggregate from the culturing method as disclosed herein enables separation in various areas of the tumor gradient microenvironment for downstream histological, cellular and molecular studies, thus positioning the device and the method as disclosed herein as a tool for evaluating tumorigenesis and tumor progression mechanisms, as well as for developing corresponding therapeutic strategies.

In another aspect, the present disclosure provides a system comprising the cell culture device as disclosed herein.

In one example, the system as disclosed herein comprises the cell culture device as disclosed herein. In another example, the system as disclosed herein comprises the cell culture device as disclosed herein and a multi-well cell culture plate. In another example, the system as disclosed herein comprises the cell culture device as disclosed herein and a detection device for detecting signals from the cells which are cultured in the cell culture device. In a preferred example, the system as disclosed herein comprises the cell culture device as disclosed herein, a multi-well cell culture plate, and a detection device for detecting signals from cells which are cultured in the cell culture device. In another example, the detection device is a fluorescene signal detection device. In another example, the detection device is a atomic force microscope In another example, the detection device is a fluorescence microscope. As described herein, FIG. 4 shows detection of fluorescence signals in cell aggregate cultured using in the cell culture device as disclosed herein, when cells are pretreated with pH sensors, pHrodo Red AM or BCECF AM, or the hypoxia sensor, Green Hypoxia Reagent (FIGS. 4A, 4B and 4C). FIG. 4D shows the use of atomic force microscopy to measure stiffness of the cell aggregate obtained from the cell culture device as disclosed herein.

In another aspect, the present disclosure provides a kit comprising the cell culture device as disclosed herein.

In one example, the kit comprises the cell culture device as disclosed herein. In another example, the kit comprises a multi-well cell culture plate. In another example, the kit comprises a cover for sealing the cell culture chamber of the cell culture device. In yet another example, the kit comprises reagents for culturing cells. In one example, the kit comprises the cell culture chamber as disclosed herein, a multi-well cell culture plate, a cover for sealing the cell culture chamber of the cell culture device and reagents for culturing cells.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present disclosure has been specifically disclosed by preferred embodiments and optional features, modification and variation of the inventions embodied therein herein disclosed may be resorted to by those skilled in the art, and that such modifications and variations are considered to be within the scope of this invention.

The present disclosure has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples.

### EXPERIMENTAL SECTION

### Cell Culture

SK-Hep-1 wide-type cell lines and SK-Hep-1 cells transfected with GFP were maintained in Dulbecco's modified Eagle's medium (DMEM, Invitrogen) supplemented with 10% fetal bovine serum (FBS, Invitrogen) and 1% penicillin-streptomycin (Invitrogen). The cells and the cell culture device loaded with cells were incubated in a humidified incubator at 37° C. with 5% CO₂.

### Design of the Cell Culture Device

The culturing chamber of the cell culture device was designed with three rows of concentric pillars. The distance between pillars on each concentric row was 100 μm. Additionally, the radius difference between rows of pillars was also 100 μm. The pillars had a cross-sectional dimension of 100 mm in length and 50 mm in width. Sparsely arranged micropillars were designed inside the inner area of the cell culture device to support its spatial structure. The mold for this top component of the cell culture device was fabricated using standard photolithographic procedures. Briefly, to generate a 300-micrometer-thick pattern on a silicon wafer, SU-8 2150 resist from MicroChem Corp. was spin-coated onto a silicon wafer at 500 rpm for 10 seconds, followed by a spin at 2000 rpm for 30 seconds. Subsequently, a soft bake, UV exposure at 250 mJ/cm² at 365 nm, and post-baking were conducted. The resulting exposed and developed SU-8 layer served as a mold for PDMS casting. The PDMS component was affixed to the glass substrate to facilitate subsequent disassembly using a thin layer of uncured PDMS. A brief incubation at 80° C. for 15 minutes enhanced the binding.

### Cell Loading Into the Cell Culture Chamber of the Cell Culture Device

In the central region of the cell culture device, an opening with a pre-defined diameter of 0.5 to 1 mm was punched. The cells were first enzymatically dissociated and centrifuged to acquire cell pellets. Cell pellets, combined with 10% GelMA- 60, were introduced into the cell culture chamber through the opening until they completely covered the region enclosed by the concentric pillars. A brief 15-second exposure to UV light was applied to solidify the cell-GelMA mixture at a power density of 60 mW/cm² at 405 nm. Following solidification of the cell pellet, the surface of the cell culture chamber was covered with a thin cover glass with a diameter of 9 mm to seal the opening and to prevent the diffusion of oxygen. The cell culture chamber was then supplemented with DMEM medium through the cell culture reservoirs located at opposite ends of the cell culture chamber and then placed in a 6-well plate and cultured under standard cell culture conditions. Notably, the gaps outside the wells of the 6-well plate should be filled with sterile water to prevent excessive evaporation of the culture medium in the cell culture device. The medium was replaced every two days to maintain optimal growth conditions.

### Characterization of pH, Hypoxia, and Stiffness Profile of the Tumor Spheroid

To assess the pH and oxygen gradients within the cell culture device, commercially available dyes, including pHrodo Red AM (P35372, Thermo Fisher) and BCECF AM (S1006,Beyotime) were used for pH analysis, and Image-iT Green Hypoxia Reagent (I14833, Thermo Fisher) was used for oxygen analysis. Following the supplier's recommendations, these compounds were dissolved in dimethyl sulfoxide (DMSO). Staining procedures were conducted according to the manufacturer's guidelines. Wild-type SK-Hep1 cells were initially trypsin-digested, washed with phosphate buffer saline (PBS), and subsequently incubated with the respective dyes. The cells marked with probes were washed once with PBS, loaded into the cell culture device as previously described above and incubated in the incubator. Cells on the cell culture device were imaged using Nikon Eclipse Ti2 inverted microscope 8 hours post-incubation. The analysis of the stiffness profile of the cells grown in the cell culture chamber was performed according to previously reported methods. In brief, on the seventh day, when the gradient microenvironment had formed and stabilized, the cell culture device was extracted from the 6-well plate, and the PDMS layer of the cell culture device was meticulously removed from the glass substrate. The cell aggregates on the glass substrate were immersed in PBS and prepared for atomic force microscopy (AFM) measurement using the Bioscope Catalyst microscope (Bruker Instruments, USA) in contact mode (see FIG. 4D). A pyramidal tip with a nominal radius of 20 nm was used. Before each measurement, each probe was calibrated for deflection sensitivity and spring constant on a glass slide. The Poisson's ratio was assumed to be 0.3, and the Sneddon model for force curve fitting was used to calculate Young's modulus.

### Diffusion Gradient Analysis

Diffusion barrier analyses were conducted based on rhodamine diffusion assessments, using three distinct operational modes to create different microenvironment. The first consisted solely of GelMA without loaded cells (Chip_GelMA), while the second was loaded with GelMA and cells (Chip_Day0) prior to culturing. The third featured a stable gradient microenvironment that was cultured for 10 days (Chip_env). Rhodamine was diluted into DMEM at a final concentration of 10 μM. The cells cultured in the device underwent three washes with Rhodamine-containing DMEM to replace the original medium. Subsequently, the device was positioned under a Nikon Eclipse Ti-E live cell imaging system for observation. For Chip_GelMA, images were captured at 10-minute intervals over a 2-hour duration. The Chip_Day 0 was monitored at 20-minute intervals throughout a 12-hour timeframe. With regard to Chip_env, the images were recorded at 10-minute intervals over 18 hours.

### Paraffin Sections and Immunofluorescence Staining Analysis

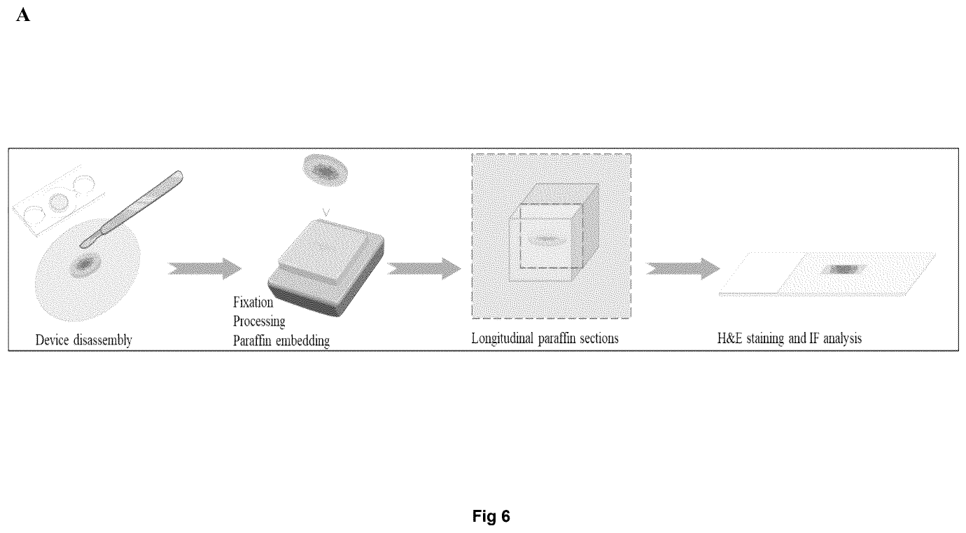

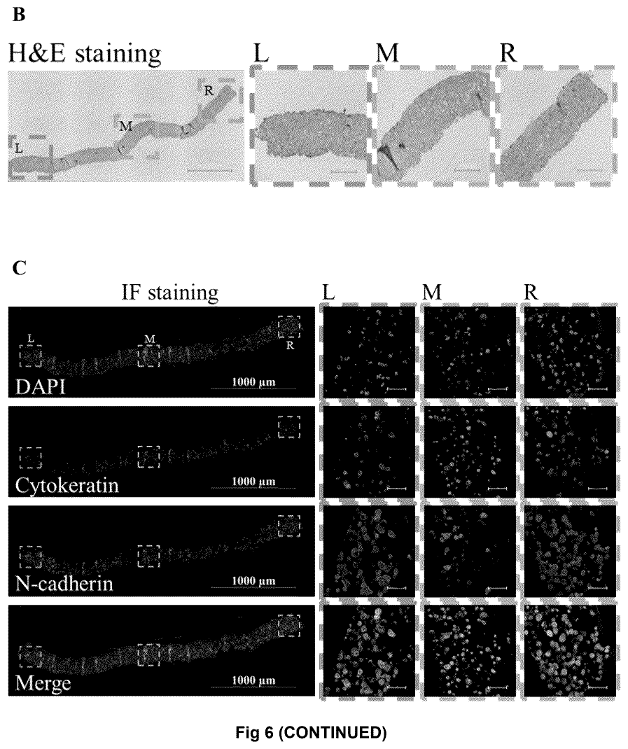

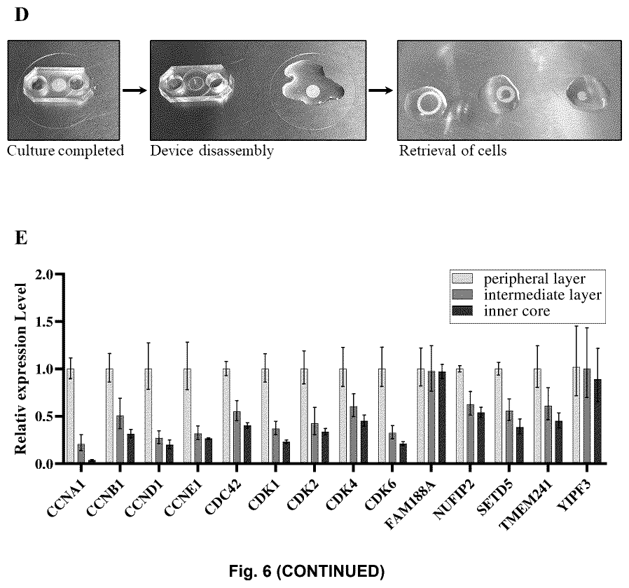

The PDMS top layer was removed from the bottom glass substrate, and then the residual cell aggregates on the glass substrate were fixed using 4% paraformaldehyde. Following fixation, a thin surgical blade was used to carefully separate the cell aggregates from the glass substrate. Subsequently, standard tissue sectioning procedures were applied on the cell aggregates (FIG. 6A). The cell aggregates section then underwent a standard Haematoxylin and Eosin (HE) staining step. Slides were then analyzed with cytokeratin monoclonal antibody (mAb), N-cadherin mAb, and DAPI. Photomicrographs represented in FIGS. 6B and 6C were captured using a Nikon Eclipse Ti2 inverted microscope.

### qPCR Analysis

In order to investigate the gene expression profiles of cells in different gradient environmental regions, a series of experimental procedures were conducted. Initially, PDMS layers were removed to expose the cell aggregates. Subsequently, the aggregates were divided into inner, middle, and outer layers using a puncher (FIG. 6D). The segregated cell aggregates from distinct regions were transferred to DMEM medium supplemented with collagenase (500 U/mL) and then incubated at 37° C. for 1 hour to release the cells form the Cell-GelMA mixture. The obtained cell pallets were washed twice with PBS and then added with 1 mL of Trizol (T9108, Takara) for standard RNA extraction. Following RNA extraction, reverse transcription was performed using PrimeScrip RT Master Mix (RR036, Takara), followed by qPCR analysis (QuantStudio 12K Flex), according to standard protocols.

### Statistical Analysis

Fluorescence intensities were quantified using the Nikon TI2E system, and data visualization was carried out employing the R and image J. Scheme 1 was generated with BioRender. com. The error bars presented in the figures represent the standard deviation (SD) derived from at least three tests. Statistical comparisons between groups were conducted using the student's t-test to assess associations between independent variables, and the corresponding p-values were determined.

### Results

### 1. Phenotype of the Spheroid-Like Gradient Microenvironment on the Cell Culture Device

SK-Hep1 cells transfected with GFP were used to demonstrate the establishment of the spheroid-like gradient microenvironment on the cell culture device. As shown in FIG. 2, culturing of the tumor cells on the cell culture device successfully replicated the gradient of cellular viability, closely resembling the classical structure of tumor spheroids. This structure comprises a peripheral region with the highest proliferation of cells, followed by an intermediate quiescent layer where cellular activity gradually decreases, culminating in the central apoptotic region, where is nearly devoid of cells (FIG. 2A, FIG. 2C and FIG. 2D). The relative size of the cell aggregates obtained from culturing cells in the cell culture device was compared to tumor spheroids obtained from culturing cells in suspension culture in a dish. The diameter of tumor spheroids obtained from cells cultured in suspension culture ranges from 100 μm to 900 μm, while the cell aggregate obtained from culturing tumor cells in the cell culture device is approximately 6 mm. Therefore, culturing of tumor cells using the cell culture device overcomes the inherent size limitation using the suspension cell culture method (FIG. 2B).

### 2. Higher Initial Cell Density Accelerates Formation of the Gradient Microenvironment

In this study, the impact of initial cell density on the establishment of spheroid-like gradient microenvironments by culturing cell in the cell culture device was established. As shown in FIG. 3A, loading the cells-GelMA mixture at a density of 1×10⁶ cells/10 μL GelMA resulted in the obvious appearance of gradient characteristics from Day 4. However, with decreased cell densities of 5×10⁵ cells/10 μL and 2.5×10⁵ cells/10 μL, gradient features only began to appear from day 6 and day 14, respectively. Cell aggregates obtained from cells grown using the cell culture device with an initial cell density lower than 2.5×10⁵ cells/10 μL struggled to form the gradient microenvironment within the monitored 14 days, resembling conventional 3D spheroid culture microenvironments. The trend in fluorescence intensity over time clearly illustrates that as cell density decreases, nutrient consumption rate gradually falls below diffusion rate, making it difficult to establish the gradient environment (FIG. 3B). The cells remaining in the central loading inlet gradually died as the gradient environment was established, having minimal impact on the final microenvironment phenotype.

### 3. Gradient Distribution Features on the Chip

Three commercial live-staining probes were employed to characterize the gradient features of the cell aggregates cultured using the cell culture device. The pH distribution status of the cells is established using pHrodo Red AM. FIG. 4A reveals a gradient increased fluorescence signal from the periphery toward the central region, which indicates that the central region has a more acidic microenvironment profile compared to the periphery. Due to the cell loading method mentioned earlier, residual cells in the loading inlet can cause a bright spot in the centre. Although this does not affect the formation of the final microenvironment, to eliminate potential non-specific interference on the results, another probe, BCECF AM, was introduced to further confirm the pH distribution profiles of the cell aggregate cultured on the cell culture device. The fluorescence generation mechanism of BCECF AM is opposite to pHrodo Red AM, wherein cells labelled with BCECF AM exhibited lower signal intensity in lower pH microenvironments. The results were consistent. Staining with BCECF AM confirmed a gradient decrease of the signal from the periphery toward the central area, validating the low pH level in the central area (FIG. 4B). Moreover, non-specific fluorescence within the cell loading inlet was limited to a very small range, and its impact on the surrounding signals can be negligible (FIG. 4C).

Next, cell staining with the hypoxia probe Image-iT Green confirmed the cells in the central area were subjected to high levels of hypoxic conditions, gradually alleviating towards the outer regions (FIG. 4C). Furthermore, the top PDMS layer could be disassembled from the cell culture device to expose the cell aggravates to characterize the stiffness distribution profiles, a feat that is difficult to achieve with traditional tumor spheroid models. After seven days of culture, once the gradient phenotype had clearly formed, cell aggregates were exposed for stiffness testing. Experiments revealed a significantly higher stiffness profile in the central region (FIG. 4D).

### 4. Real-Time Monitoring of Penetration Process

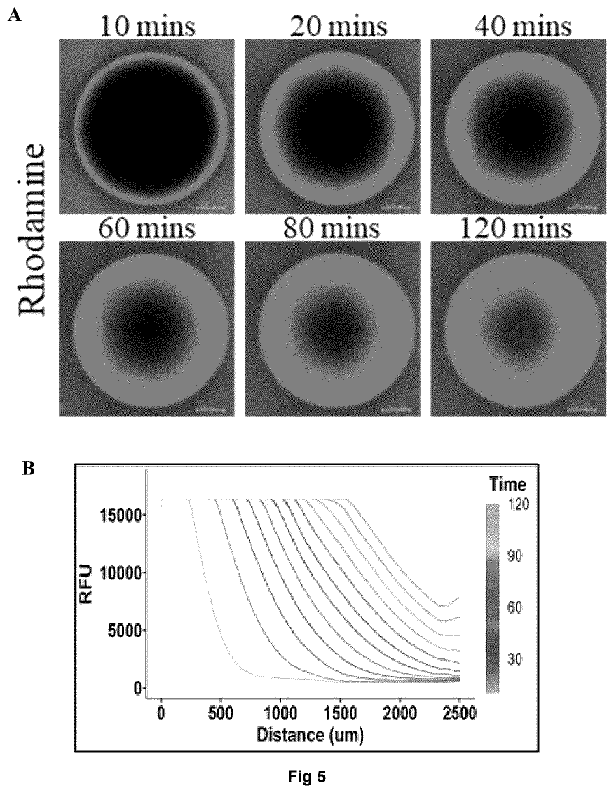

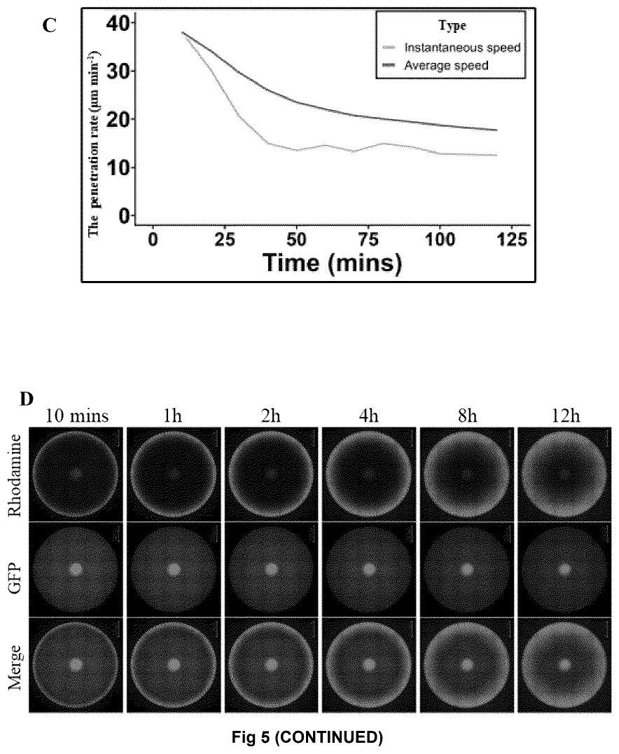

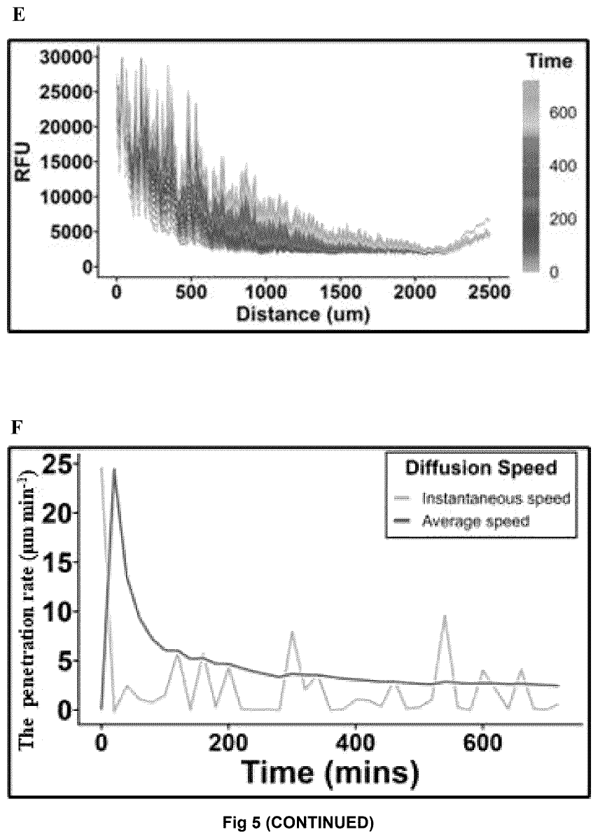

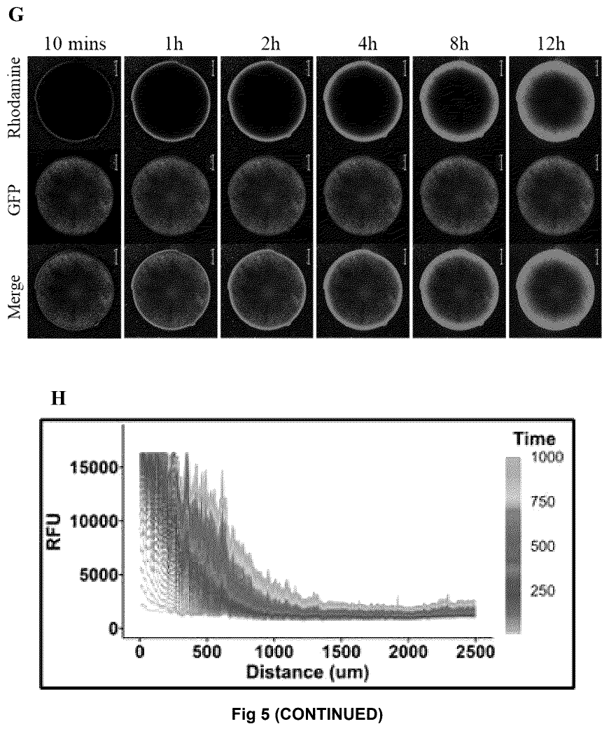

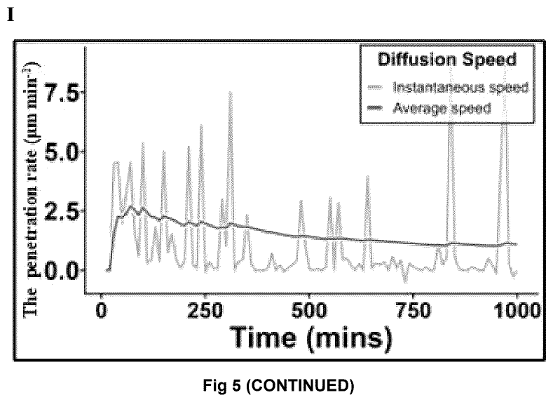

The glass substrate and circular design of the cell culture chamber facilitated real-time observation of penetration processes within the gradient microenvironment established in the cell aggregate. In this experiment, rhodamine was used to characterize its penetration ability across three distinct microenvironments. Specifically, the first microenvironment, “Chip_gel” refers to the cell culture device loaded only with GelMA without cells. The second microenvironment, “Chip_day0” represents the cell culture device that was loaded with GelMA-cell mixture prior to in vitro culture, and the third microenvironment, “Chip_env” refers to the cell culture device loaded with GelMA-cell mixture cultured for 10 days, thus represents a fully established gradient microenvironment. The penetration rate of rhodamine on Chip_gel was first assessed. Effortless permeation of Rhodamine was seen in the internal regions of the cell culture chamber within approximately 2 hours (FIGS. 5A and 5B), with a mean diffusion speed of approximately 20.7±7.3 μm/min (FIG. 5C). Subsequently, the penetration profile of rhodamine on Chip_gel was assessed. In this situation, the tumor microenvironment within the device had not yet been established. In the presence of cells, the penetration rate of rhodamine was significantly decreased (FIGS. 5D and 5E), with a penetration speed of around 4.0±2.4 μm/min (FIG. 4F). Furthermore, the introduction of rhodamine into the chip with a pre-formed gradient microenvironment revealed a prolonged penetration duration, even after an extended period exceeding 24 hours. External rhodamine struggled to penetrate into the central regions (FIGS. 5G and 5H), with an overall diffusion speed of significantly decreased to 1.53±0.2 μm/min (FIG. 5I). Towards the end of the monitoring period, the penetration rate had nearly reached zero.

### 5. Release of Cell Aggregates for Downstream Molecular Analysis

The device's disassembly capability and the retrieval of cells from different regions greatly facilitate in-depth molecular mechanism analysis. After cultivation, the PDMS layer can be easily separated from the glass substrate. Cell aggregates released from the substrate are suitable for downstream mechanistic analysis, including histological sectioning and Haematoxylin and Eosin (H&E) staining (FIGS. 6A and 6B). Staining of two EMT-related markers confirmed successful immunofluorescence staining (FIG. 6C). To further explore the device's application prospects, punches were used to segment released cell aggregates, easily obtaining cells from the inner, middle, and outer regions, each containing different gradient environment information (FIG. 6D). Subsequently, cells from segmented aggregates were released using collagenase for qPCR analysis. A noticeable decrease in expression of cell cycle related genes from the outer to inner regions, aligning with previous research indicating cellular arrest status in quiescent and necrotic areas.

1. A cell culture device comprising a top component,
wherein the top component comprises a cell culture chamber and one or more cell culture medium reservoirs;
wherein the cell culture chamber comprises an outer area and an inner area,
wherein the outer area and inner area of the cell culture chamber are separated from each other by at least one row of micropillars, and wherein the outer area of the cell culture chamber is fluidly connected to the one or more cell culture medium reservoirs, and
wherein the cell culture chamber comprises an inlet which allows addition of a mixture comprising cells to the inner area of the cell culture chamber.

2. The cell culture device of claim 1, wherein the device further comprises a bottom substrate.

3. The cell culture device of claim 2, wherein the top component of the cell culture device is detachably attachable to bottom substrate.

4. The cell culture device of claim 3, wherein the top component and the bottom substrate are bonded together.

5. The cell culture device of claim 3, wherein the cell culture device has dimensions which allow placement of the cell culture device in a multi-well cell culture plate.

6. The cell culture device of claim 5, wherein any one or both of the cell culture chamber and the one or more cell culture medium reservoirs are circular.

7. The cell culture device of claim 3, wherein the inlet is on one end of a channel connected to the inner area of the cell culture chamber on the other end of the channel.

8. The cell culture device of claim 7, wherein the channel is in the center of the inner area of the cell culture chamber.

9. The cell culture device of claim 3, wherein the size of the space between adjacent micropillars is selected to prevent leakage of the mixture comprising cells during initial loading and to allow access of the cell culture medium.

10. The cell culture device of claim 3, wherein the cell culture chamber comprises at least two rows of micropillars and wherein the micropillars between the different rows are in a staggered arrangement.

11. The cell culture device of claim 3 comprising two cell culture medium reservoirs.

12. The cell culture device of claim 11, wherein the two cell culture medium reservoirs are fluidically connected to opposite ends of the outer area of the cell culture chamber.

13. The cell culture device of claim 3, further comprising micropillars inside the inner area of the cell culture chamber.

14. A method of culturing cells using the cell culture device of claim 3.

15. The method of claim 14, wherein the method comprises the steps of:
a. loading a mixture comprising cells and a scaffold into the inner area of the cell culture chamber of the cell culture device of claim 3 through the inlet until the mixture covers the inner area of the cell culture chamber enclosed by the micropillars;
b. adding a cell culture medium to the one or more cell culture medium reservoirs;
c. sealing the inlet of the cell culture chamber loaded with the mixture with a cover; and
d. culturing the cells to obtain a cell aggregate in the inner area of the cell culture chamber.

16. The method of claim 15, wherein the mixture comprises tumor cells.

17. The method of claim 16, wherein the mixture further comprises other cell populations selected from the group consisting of stromal cells, fibroblasts and immune cells, and a combination thereof.

18. The method of claim 15, wherein the scaffold is selected from the group consisting of alginate, collagen, gelatin, gelatin methacrylate, hydrogels and polycaprolactone, and a combination thereof.

19. The method of claim 18, wherein the scaffold is gelatin methacrylate.

20. A system comprising:
a. the cell culture device of claim 3;
b. a multi-well cell culture plate; and
c. a detection device for detecting signals emitted from cells cultured in the cell culture device.
